SEQUENCE LISTING (56 sequences)

SEQ ID NO: 1 - DNA, 8185 bp, Xenotropic murine lukemia virus related virus
gcgccagtcatccgatagactgagtcgcccgggtacccgtgttcccaataaagccttttgctgtttgcatccgaagcgtggcctcgctgttccttgggagggtctcctcagagtgattgactacccagctcgggggtctttcatttgggggctcgtccgggattcggagacccccgcccagggaccaccgacccaccgtcgggaggtaagccggccggcgatcgttttgtctttgtctctgtctttgtgcgtgtgtgtgtgtgccggcatctaatcctcgcgcctgcgtctgaatctgtactagttagctaactagatctgtatctggcggttccgcggaagaactgacgagttcgtattcccggccgcagccctgggagacgtcccagcggcctcgggggcccgttttgtggcccattctgtatcagttaacctacccgagtcggactttttggagtggctttgttgggggacgagagacagagacacttcccgcccccgtctgaatttttgctttcggttttacgccgaaaccgcgccgcgcgtctgatttgttttgttgttcttctgttcttcgttagttttcttctgtctttaagtgttctcgagatcatgggacagaccgtaactacccctctgagtctaaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccctctataaagtccaaacctcctaagccccaggttctccctgatagcggcggacctctcattgaccttctcacagaggatcccccgccgtacggagcacaaccttcctcctctgccagggagaacaatgaagaagaggcggccaccacctccgaggtttcccccccttctcccatggtgtctcgactgcggggaaggagagaccctcccgcagcggactccaccacctcccaggcattcccactccgcatggggggagatggccagcttcagtactggccgttttcctcctctgatttatataattggaaaaataataacccttccttttctgaagatccaggtaaattgacggccttgattgagtccgtcctcatcacccaccagcccacctgggacgactgtcagcagttgttggggaccctgctgaccggagaagaaaagcagcgggtgctcctagaggctagaaaggcagtccggggcaatgatggacgccccactcagttgcctaatgaagtcaatgctgcttttccccttgagcgccccgattgggattacaccactacagaaggtaggaaccacctagtcctctaccgccagttgctcttagcgggtctccaaaacgcgggcaggagccccaccaatttggccaaggtaaaagggataacccagggacctaatgagtctccctcagcctttttagagagactcaaggaggcctatcgcaggtacactccttatgaccctgaggacccagggcaagaaaccaatgtgtccatgtcattcatctggcagtctgccccggatatcggacgaaagttagagcggttagaagatttaaagagcaagaccttaggagacttagtgagggaagctgaaaagatctttaataagcgagaaaccccggaagaaagagaggaacgtatcaggagagaaatagaggaaaaagaagaacgccgtagggcagaggatgagcagagagagagagaaagggaccgcagaagacatagagagatgagcaagctcttggccactgtagttattggtcagagacaggatagacaggggggagagcggaggaggccccaacttgataaggaccaatgcgcctactgcaaagaaaagggacactgggctaaggactgcccaaagaagccacgagggccccgaggaccgaggccccagacctccctcctgaccttaggtgactagggaggtcagggtcaggagcccccccctgaacccaggataaccctcaaagtcggggggcaacccgtcaccttcctggtagatactggggcccaacactccgtgctgacccaaaatcctggacccctaagtgacaagtctgcctgggtccaaggggctactggaggaaagcggtatcgctggaccacggatcgcaaagtacatctggctaccggtaaggtcacccactctttcctccatgtaccagactgcccctatcctctgctaggaagagacttgctgactaaactaaaagcccaaatccactttgagggatcaggagctcaggttgtgggaccgatgggacagcccctgcaagtgctgacagtaaacatagaagatgagtattggctacatgataccaggaaagagccagatgttcctctagggtccacatggctttctgatttccttcaggcctgggcggaaaccgggggcatgggactggcagttcgccaagctcctctgatcatacctctgaaggcaacctctacccccgtgtccataaaacaataccccatgtcacaagaagccagactggggatcaagccccacatacagaggctgttggaccagggaatactggtaccctgccagtccccctggaacacgcccctgctacccgttaagaaaccagggactaatgattataggcctgtccaggatctgagagaagtcaacaagcgggtggaagacatccaccccaccgtgcccaacccttacaacctcttgagcgggctcccaccgtcccaccagtggtacactgtgcttgatttaaaggatgcctttttctgcctgagactccaccccaccagtcagcctctcttcgcctttgagtggagagatccagagatgggaatctcaggacaactgacctggaccagactcccacagggtttcaaaaacagtcccaccctgtttgatgaggcactgcacagagacctagcagatttccggatccagcacccagacttgatcctgctacagtacgtggatgacttactgctggccgccacttctgagcaagactgccaacgaggtactcgggccctattacaaaccctagggaacctcgggtatcgggcctcggccaagaaagcccaaatttgccagaaacaggtcaagtatctggggtatctcctaaaagagggacagagatggctgactgaggccagaaaagagactgtgatggggcagcccactccgaagacccctcgacaactaagggagttcctagggacggcaggcttctgtcgcctctggatccctgggtttgcagaaatggcagcccccttgtaccctcttaccaaaacggggactctgtttaattggggcccagaccagcaaaaggcctatcaagaaatcaaacaggctcttctaactgcccccgccctgggattgccagatttgactaagccctttgaactctttgtcgacgagaagcagggctacgccaaaggcgtcctaacgcaaaaactgggaccttggcgtcggcctgtggcctacctgtccaaaaagctagacccagtggcagctgggtggcccccttgcctacggatggtagcagccattgccgttctgacaaaaaatgcaggcaagctaactatgggacagccgctagtcattctggccccccatgcggtagaagcactggtcaaacaaccccctgaccgttggctatccaatgcccgcatgacccactatcaggcaatgctcctggatacagaccgggttcagttcggaccggtggtggccctcaacccggccaccctgctccccctaccggaaaaggaagccccccatgactgcctcgagatcttggctgagacgcacggaaccagaccggacctcacggaccagcccatcccagacgctgattacacttggtacacagatggaagcagcttcctacaagaaggacaacggagagctggagcagcggtgactactgagaccgaggtaatctgggcgagggctctgccggctggaacatccgcccaacgagccgaactgatagcactcacccaagccttaaagatggcagaaggtaagaagctaaatgtttacactgatagccgctatgccttcgccacggcccatgtccatggagaaatatataggaggcgagggttgctgacctcagaaggcagagaaattaaaaacaagaacgagatcttggccttgctaaaagctctctttctgcccaaacgacttagtataattcactgtccaggacatcaaaaaggaaacagtgctgaggccagaggcaaccgtatggcagatcaagcagcccgagaggcagccatgaaggcagttctagaaacctctacactcctcatagaggactcaaccccgtatacgcctccccatttccattacaccgaaacagatctcaaaagactacgggaactgggagccacctacaatcagacaaaaggatattgggtcctacaaggcaaacctgtgatgcccgatcagtccgtgtttgaactgttagactccctacacagactcacccatctgagccctcaaaagatgaaggcactcctcgacagagaagaaagcccctactacatgttaaaccgggacagaactatccagtatgtgactgagacctgcaccgcctgtgcccaagtaaatgccagcaaagccaaaattggggcaggggtgcgagtacgcggacatcggccaggcacccattgggaagttgatttcacggaagtaaagccaggactgtatgggtacaagtacctcctagtgtttgtagacaccttctctggctgggtagaggcattcccgaccaagcgggaaactgccaaggtcgtgtccaaaaagctgttagaagacatttttccgagatttggaatgccgcaggtattgggatctgataacgggcctgccttcgcctcccaggtaagtcagtcagtggccgatttactggggatcgattggaagttacattgtgcttatagaccccagagttcaggacaggtagaaagaatgaatagaacaattaaggagactttgaccaaattaacgcttgcatctggcactagagactgggtactcctactccccttagccctctaccgagcccggaatactccgggcccccacggactgactccgtatgaaattctgtatggggcacccccgccccttgtcaattttcatgatcctgaaatgtcaaagttaactaatagtccctctctccaagctcacttacaggccctccaagcagtacaacaagaggtctggaagccgctggccgctgcttatcaggaccagctagatcagccagtgataccacaccccttccgtgtcggtgacgccgtgtgggtacgccggcaccagactaagaacttagaacctcgctggaaaggaccctacaccgtcctgctgacaacccccaccgctctcaaagtagacggcatctctgcgtggatacacgccgctcacgtaaaggcggcgacaactcctccggccggaacagcatggaaagtccagcgttctcaaaaccccttaaagataagattaacccgtggggccccctgataattatggggatcttggtgagggcaggagcctcagtacaacgtgacagccctcaccaggtctttaatgtcacttggaaaattaccaacctaatgacaggacaaacagctaatgctacctccctcctggggacgatgacagacactttccctaaactatattttgacttgtgtgatttagttggagacaactgggatgacccggaacccgatattggagatggttgccgctctcccgggggaagaaaaaggacaagactatatgatttctatgtttgccccggtcatactgtattaacagggtgtggagggccgagagagggctactgtggcaaatggggatgtgagaccactggacaggcatactggaagccatcatcatcatgggacctaatttcccttaagcgaggaaacactcctaagggtcagggcccctgttttgattcctcagtgggctccggtagcatccagggtgccacaccggggggtcgatgcaaccccctagtcctagaattcactgacgcgggtaaaagggccagctgggatgcccccaaaacatggggactaagactgtatcgatccactggggccgacccggtgaccctgttctctctgacccgccaggtcctcaatgtagggccccgcgtccccattgggcctaatcccgtgatcactgaacagctacccccctcccaacccgtgcagatcatgctccccaggactcctcgtcctcctccttcaggcgcggcctctatggtgcctggggctcccccgccttctcaacaacctgggacgggagacaggctgctaaacctggtagaaggagcctacctagccctcaacctcaccagtcccgacaaaacccaagagtgctggctgtgtctagtatcgggacccccctactacgaaggggtggccgtcctaggtacttactccaaccatacctctgccccggctaactgctccgtgacctcccaacacaagctgaccctgtccgaagtgaccgggcagggactctgcataggagcagttcccaaaacccatcaggccctgtgtaataccacccagaagacgagcgacgggtcctactatttggcctctcccgccgggaccatttgggcttgcagcaccgggctcactccctgtctatctactactgtgcttaacttaaccactgattactgtgtcctggttgaactctggccaaaggtaacctaccactcccctaattatgtttatggccagtttgaaaagaaaactaaatataaaagagagccggtgtcattaactctggccctgctgttgggaggacttactatgggcggcatagctgcaggagttggaacagggactacagccctagtggccaccaaacaattcgagcagctccaggcagccatacatacagaccttggggccttagaaaaatcagtcagtgccctagaaaagtctctgacctcgttgtctgaggtggtcctacagaaccggaggggattagatctactgttcctaaaagaaggaggattatgtgctgccctaaaagaagaatgctgtttttacgcggaccacactggcgtagtaagagatagcatggcaaagctaagagaaaggttaaaccagagacaaaaattgttcgaatcaggacaagggtggtttgagggactgtttaacaggtccccatggttcacgaccctgatatccaccattatgggccctctgatagtacttttattaatcctactcttcggaccctgtattctcaaccgcttggtccagtttgtaaaagacagaatttcggtagtgcaggccctggttctgacccaacagtatcaccaactcaaatcaatagatccagaagaagtggaatcacgtgaataaaagattttattcagtttccagaaagaggggggaatgaaagaccccaccataaggcttagcacgctagctacagtaacgccattttgcaaggcatggaaaagtaccagagctgagttctcaaaagttacaaggaagtttaattaaagaataaggctgaataacactgggacaggggccaaacaggatatctgtagtcaggcacctgggccccggctcagggccaagaacagatggtcctcagataaagcgaaactaacaacagtttctggaaagtcccacctcagtttcaagttccccaaaagaccgggaaataccccaagccttatttaaactaaccaatcagctcgcttctcgcttctgtacccgcgctttttgctccccagtcctagccctataaaaaaggggtaagaactccacactcggcgcgccagtcatccgatagactgagtcgcccgggtacccgtgttcccaataaagccttttgctgtttgcaa

SEQ ID NO: 2 - protein, 1733 aa, Xenotropic murine lukemia virus related virus
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTVNIEDEYWLHDTRKEPDVPLGSTWLSDFLQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKNAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 3 - protein, 536 aa, Xenotropic murine lukemia virus related virus
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGD

SEQ ID NO: 4 - protein, 645 aa, Xenotropic murine lukemia virus related virus
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRTPRPPPSGAASMVPGAPPPSQQPGTGDRLLNLVEGAYLALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 5 - DNA, 25 bp, Artificial Sequence
  419F PCR primer
atcagttaacctacccgagtcggac

SEQ ID NO: 6 - DNA, 23 bp, Artificial Sequence
  1154R PCR primer
gccgcctcttcttcattgttctc

SEQ ID NO: 7 - DNA, 22 bp, Artificial Sequence
  5922F PCR primer
gctaatgctacctccctcctgg

SEQ ID NO: 8 - DNA, 26 bp, Artificial Sequence
  6273R PCR primer
ggagcccactgaggaatcaaaacagg

SEQ ID NO: 9 - DNA, 25 bp, Artificial Sequence
  4F PCR primer
ccagtcatccgatagactgagtcgc

SEQ ID NO: 10 - DNA, 26 bp, Artificial Sequence
  770R PCR primer
taccatcctgaggccatcctacattg

SEQ ID NO: 11 - DNA, 23 bp, Artificial Sequence
  350F PCR primer
gagttcgtattcccggccgcagc

SEQ ID NO: 12 - DNA, 21 bp, Artificial Sequence
  5135R PCR primer
cctgcggcattccaaatctcg

SEQ ID NO: 13 - DNA, 25 bp, Artificial Sequence
  4789R PCR primer
gggtgagtctgtgtagggagtctaa

SEQ ID NO: 14 - DNA, 25 bp, Artificial Sequence
  4166F PCR primer
caagaaggacaacggagagctggag

SEQ ID NO: 15 - DNA, 24 bp, Artificial Sequence
  7622R PCR primer
ggcctgcactaccgaaattctgtc

SEQ ID NO: 16 - DNA, 27 bp, Artificial Sequence
  4672F PCR primer
gagccacctacaatcagacaaaaggat

SEQ ID NO: 17 - DNA, 25 bp, Artificial Sequence
  7590R PCR primer
ctggaccaagcggttgagaatacag

SEQ ID NO: 18 - DNA, 21 bp, Artificial Sequence
  7472F PCR primer
tcaggacaagggtggtttgag

SEQ ID NO: 19 - DNA, 23 bp, Artificial Sequence
  8182R PCR primer
caaacagcaaaaggctttattgg

SEQ ID NO: 20 - DNA, 19 bp, Artificial Sequence
  8147R PCR primer
ccgggcgactcagtctatc

SEQ ID NO: 21 - DNA, 21 bp, Artificial Sequence
  MCox2-F2 PCR primer
ttctaccagctgtaatcctta

SEQ ID NO: 22 - DNA, 22 bp, Artificial Sequence
  MCox2-R1 PCR primer
gttttaggtcgtttgttgggat

SEQ ID NO: 23 - DNA, 31 bp, Artificial Sequence
  MCox2-PR1 PCR primer
cgtagcttcagtatcattggtgccctatggt

SEQ ID NO: 24 - DNA, 26 bp, Artificial Sequence
  MCox2-P1 PCR primer
ttgctctcccctctctacgcattcta

SEQ ID NO: 25 - DNA, 377 bp, Xenotropic murine lukemia virus related virus
  misc_feature (3)..(3): n is a, c, g, or t
ccngattaccttgcagcactggggagatgtccagcgcattgcatccaaccagtctgtggatgtcaggaagaggcgctggattaccttctgttccgctgaatggccaactttcaatgtgggatggcctcaggatggtactttcaatttaagtattatctctcaggttaagtctagagtgttttgtcctggtccccacggacacccggatcaggtcccatatatcgtcacctgggaggcacttgcctatgacccccccgtgggtcaaaccgtttgtgtctcctaaacttcctcccttgccgacagctcccgtcctcccgcccggtccttctgcgcaacctccgtcccgatctgccctttaccctgcccttaccctctaa

SEQ ID NO: 26 - DNA, 384 bp, Xenotropic murine lukemia virus related virus
  misc_feature (4)..(4): n is a, c, g, or t
ctcngatctaccttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgcccaatacactgcccttacaaaaataaaa

SEQ ID NO: 27 - DNA, 366 bp, Xenotropic murine lukemia virus related virus
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccc

SEQ ID NO: 28 - DNA, 366 bp, Xenotropic murine lukemia virus related virus
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccctc

SEQ ID NO: 29 - DNA, 366 bp, Xenotropic murine lukemia virus related virus
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtattatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccccc

SEQ ID NO: 30 - DNA, 366 bp, Xenotropic murine lukemia virus related virus
  misc_feature (364)..(365): n is a, c, g, or t
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgccctttaccctgcccttaccnna

SEQ ID NO: 31 - DNA, 366 bp, Xenotropic murine lukemia virus related virus; identical to SEQ ID NO: 28

SEQ ID NO: 32 - DNA, 363 bp, Xenotropic murine lukemia virus related virus
ttgcagcactggggagatgtccagcgcattgcatccaaccagtctgtggatgtcaagaagaggcgctgggttaccttctgttccgccgaatggccaactttcaatgtaggatggcctcaggatggtacttttaatttaggtgttatctctcaggtcaagtctagagtgttttgtcctggtccccacggacacccggatcaggtcccatatatcgtcacctgggaggcacttgcctatgacccccctccgtgggtcaaaccgtttgtctctcctaaaccccctcctttaccgacagctcccgtcctcccgcccggtccttctgcgcaacctccgtcccgatctgcccaatacactgcccttaca

SEQ ID NO: 33 - protein, 645 aa, Xenotropic MuLV-related Virus VP35
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFGKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKKECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 34 - protein, 1733 aa, Xenotropic MuLV-related Virus VP35
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTLSIKSKPPKPQVLPDSGGPLIDLLTEDPPPYGVQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEAGKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIENKYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHPSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVTKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 35 - protein, 536 aa, Xenotropic MuLV-related Virus VP35
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTLSIKSKPPKPQVLPDSGGPLIDLLTEDPPPYGVQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEAGKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGD

SEQ ID NO: 36 - protein, 645 aa, Xenotropic MuLV-related Virus VP42
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESGQGWFEGLFNRSPWFTTLISTIMGPLIVLLLILLFGPCILNRLVQFVKDRISVVQALVLTQQYHQLKSIDPEEVESRE

SEQ ID NO: 37 - protein, 1733 aa, Xenotropic MuLV-related Virus VP42
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGIISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWGYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGGSFLQEGQRRAGAAVTTETEVIWGGVLPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 38 - protein, 536 aa, Xenotropic MuLV-related Virus VP42
MGQTVTTPLSLTLQHWGDVQRIASNQSVDVKKRRWVTFCSAEWPTFNVGWPQDGTFNLGIISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWGYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGD

SEQ ID NO: 39 - protein, 645 aa, Xenotropic MuLV-related Virus VP62; identical to SEQ ID NO: 4

SEQ ID NO: 40 - protein, 1733 aa, Xenotropic MuLV-related Virus VP62; identical to SEQ ID NO: 2
  misc_feature (537)..(537): Xaa can be any naturally occurring amino acid

SEQ ID NO: 41 - protein, 536 aa, Xenotropic MuLV-related Virus VP62; identical to SEQ ID NO: 3

SEQ ID NO: 42 - protein, 645 aa, Xenotropic MuLV-related Virus VP62
MESPAFSKPLKDKINPWGPLIIMGILVRAGASVQRDSPHQVFNVTWKITNLMTGQTANATSLLGTMTDTFPKLYFDLCDLVGDNWDDPEPDIGDGCRSPGGRKRTRLYDFYVCPGHTVLTGCGGPREGYCGKWGCETTGQAYWKPSSSWDLISLKRGNTPKGQGPCFDSSVGSGSIQGATPGGRCNPLVLEFTDAGKRASWDAPKTWGLRLYRSTGADPVTLFSLTRQVLNVGPRVPIGPNPVITEQLPPSQPVQIMLPRPPRPPPSGAASMVPGAPPPSQQPGTGDRLLNLVEGAYQALNLTSPDKTQECWLCLVSGPPYYEGVAVLGTYSNHTSAPANCSVTSQHKLTLSEVTGQGLCIGAVPKTHQALCNTTQKTSDGSYYLASPAGTIWACSTGLTPCLSTTVLNLTTDYCVLVELWPKVTYHSPNYVYGQFEKKTKYKREPVSLTLALLLGGLTMGGIAAGVGTGTTALVATKQFEQLQAAIHTDLGALEKSVSALEKSLTSLSEVVLQNRRGLDLLFLKEGGLCAALKEECCFYADHTGVVRDSMAKLRERLNQRQKLFESRQGWFEGLFNRSPWFTTLISTIMGPLIVLLLILLFGPCILNRLVQFVKDRISVVQALVLTQQYHQLKSIDPEEVESRE

SEQ ID NO: 43 - protein, 1733 aa, Xenotropic MuLV-related Virus VP62
  misc_feature (537)..(537): Xaa can be any naturally occurring amino acid
MGQTVTTPLSLTLQHWGDVQRIASNQSVDVKKRRWVTFCSAEWPTFNVGWPQDGTFNLGVISQVKSRVFCPGPHGHPDQVPYIVTWEALAYDPPPWVKPFVSPKPPPLPTAPVLPPGPSAQPPSRSALYPALTPSIKSKPPKPQVLPDSGGPLIDLLTEDPPPYGAQPSSSARENNEEEAATTSEVSPPSPMVSRLRGRRDPPAADSTTSQAFPLRMGGDGQLQYWPFSSSDLYNWKNNNPSFSEDPGKLTALIESVLITHQPTWDDCQQLLGTLLTGEEKQRVLLEARKAVRGNDGRPTQLPNEVNAAFPLERPDWDYTTTEGRNHLVLYRQLLLAGLQNAGRSPTNLAKVKGITQGPNESPSAFLERLKEAYRRYTPYDPEDPGQETNVSMSFIWQSAPDIGRKLERLEDLKSKTLGDLVREAEKIFNKRETPEEREERIRREIEEKEERRRAEDEQRERERDRRRHREMSKLLATVVIGQRQDRQGGERRRPQLDKDQCAYCKEKGHWAKDCPKKPRGPRGPRPQTSLLTLGDXGGQGQEPPPEPRITLKVGGQPVTFLVDTGAQHSVLTQNPGPLSDKSAWVQGATGGKRYRWTTDRKVHLATGKVTHSFLHVPDCPYPLLGRDLLTKLKAQIHFEGSGAQVVGPMGQPLQVLTLNIEDEYRLHETSKEPDVPLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQAMLLDTDRVQFGPVVALNPATLLPLPEKEAPHDCLEILAETHGTRPDLTDQPIPDADYTWYTDGSSFLQEGQRRAGAAVTTETEVIWARALPAGTSAQRAELIALTQALKMAEGKKLNVYTDSRYAFATAHVHGEIYRRRGLLTSEGREIKNKNEILALLKALFLPKRLSIIHCPGHQKGNSAEARGNRMADQAAREAAMKAVLETSTLLIEDSTPYTPPHFHYTETDLKRLRELGATYNQTKGYWVLQGKPVMPDQSVFELLDSLHRLTHLSPQKMKALLDREESPYYMLNRDRTIQYVTETCTACAQVNASKAKIGAGVRVRGHRPGTHWEVDFTEVKPGLYGYKYLLVFVDTFSGWVEAFPTKRETAKVVSKKLLEDIFPRFGMPQVLGSDNGPAFASQVSQSVADLLGIDWKLHCAYRPQSSGQVERMNRTIKETLTKLTLASGTRDWVLLLPLALYRARNTPGPHGLTPYEILYGAPPPLVNFHDPEMSKLTNSPSLQAHLQALQAVQQEVWKPLAAAYQDQLDQPVIPHPFRVGDAVWVRRHQTKNLEPRWKGPYTVLLTTPTALKVDGISAWIHAAHVKAATTPPAGTAWKVQRSQNPLKIRLTRGAP

SEQ ID NO: 44 - protein, 536 aa, Xenotropic MuLV-related Virus VP62; identical to SEQ ID NO: 3

SEQ ID NO: 45 - protein, 645 aa, Xenotropic MuLV-related Virus VP62; identical to SEQ ID NO: 4

SEQ ID NO: 46 - protein, 1733 aa, Xenotropic MuLV-related Virus VP62; identical to SEQ ID NO: 2
  misc_feature (537)..(537): Xaa can be any naturally occurring amino acid

SEQ ID NO: 47 - protein, 536 aa, Xenotropic MuLV-related Virus VP62; identical to SEQ ID NO: 3

SEQ ID NO: 48 - protein, 409 aa, Friend Spleen Focus-Forming Virus (isolate 502)
MKGPAFSKPLKDKINPWGPLIVLGILIRAGVSVQHDSPHQVFNVTWRVTNLMTGQTANATSLLGTMTDAFPMLHFDLCDLIGDDWDETGLECRTPGGRKRARTFDFYVCPGHTVPTGCGGPREGYCGKWGCETTGQAYWKPSSSWDLISLKRGNTPKDRGPCYDSSVSSGVQGATPGGRCNPLVLKFTDAGKKASWDSPKVWGLRLYRPTGIDPVTRFSLTRQVLNIGPRIPIGPNPVIIGQLPPSRPVQVRLPRPPQPPPTGAASMVPGTAPPSQQPGTGDRLLNLVQGAYQALNLTNPDKTQECWLCLVSGPPYYEGVAVLGTNSNHTSALKEKCCFYADHTGLVRDSMAKLRKRLTQRQKLFESSQGWFEGSFNRSPWFTTLISTIMGLLIILLLLLILLLWTLHS

SEQ ID NO: 49 - protein, 187 aa, Friend Spleen Focus-Forming Virus (isolate 502)
MGQTVTTPLSLTLEHWEDVQRTASNQSVDVKKRRWVTFCSAEWPTFGVGWPQDGTFNLDIILQVKSKVFSPGPHGHPDQVPYIVTWEAIAYEPPPWVKPFVSPKLSPSPTAPILPSGPSTQPPPRSALYPALTPSIKPGPSPIMADLSLTFSQKTLRRTEDRDRPPLTEMATEKRPPPLLRFLPPLP

SEQ ID NO: 50 - protein, 356 aa, Friend Spleen Focus-Forming Virus (strain BB6)
MEGPAFSKPLKDKINPWGPLIVLGILIRAGVSVQRDSPHQVFNVTWRVTNLMTGQTANATSLLGTMTDAFPKLYFDLCDLIGNDWDETRLGCRTPGEGKRARTFDLYVCPGHTVPTGCGGPREGYCGKWGCETTGQAYWKPSSSWDLISLKRGNTPKDRGPCYDSSVSSGVQGATPGGRCNPLVLKFTDAGKKASWDAPKVWGLRLYRSTGTDPVTRFSLTRQVLNIGPRVPIGPNPVISDQLPPSRPAQIMLPRPPQPPPPGTASIVPETAPPSQQPGTRDRLLNLVNKAYQALNLTSPDKTQECWLCLVSRPPYYEGVAVLGTNSNHTTLISTIMGLLIILLLLLILLLWTLHS

SEQ ID NO: 51 - protein, 409 aa, Friend Spleen Focus-Forming Virus (strain Lilly-Steeves)
MEGPASSKPLKDKTNPWGPLIILGILIRAGVSVQLDSPHQVSNVTWRVTNLMTGQTANATSLLGTMTEAFPKLYFDLCDLMGDDWDETGLGCRTPGGRKRARTFDFYVCPGHTVPTGCGGPREGYCGKWGCETTGQAYWKPSSSWDLISLKRGNTPKDQGPCYDSSVSSGVLGATPGGRCNPLVLEFTDAGRKASWDAPKVWGLRLYRSTGTDPVTRFSLTRQVLDIGPRVPIGSNPVTTDQLPLSRPVQTMPPRPLQPPPPGAASIVPETAPPPQQPGAGDRLLNLVDGAYQALNLTNPDKIQECWLCLVSGPPYYEGVVVLGTYFNHTIALKEKCCFYADHTGLVRDSMAKLRKRLTQRQKLFESSRGWFEGSSNRSPWFTTLISAIMGSLIILLLLLILLIWTLYS

SEQ ID NO: 52 - protein, 408 aa, Rauscher Spleen Focus-Forming Virus
MEGPAFSKPLKDKINPWGPLIILGILIRAGVSVQHDSPHQVFNVTWRVTNLMTGQTANATSLLGTMTDAFPKLYFDLCDLIGDDWDETGLGCRTPGGRKRARTFDFYVCPGHTVPTGCGGPREGYCGKWGCETTGQAYWKPSSSWDLISLKRGNTPRNQGPCYDSSAVSSDIKGATPGGRCNPLVLEFTDAGKKASWDGPKVWGLRLYRSTGTDPVTRFSLTRQVLNIGPRVPIGPNPVITDQLPPSRPVQIMLPRPPQPPPPGAASIVPETAPPSQQPGTGDRLLNLVDGAYQALNLTNPDKTQDCWLCLVSGPPYYEGVAVLGTYYNHTSALKEECCFYADHTGLVRDSMAKLRERLTQRQKLFESSQGWFEELFNRSTWFTTLIFTIIGPLIILLLILLFWTLHS

SEQ ID NO: 53 - protein, 116 aa, Rauscher Spleen Focus-Forming Virus
AHLHALYLVHHEVWRPLAAAYQHQLDRPIVPHPFRLGDTVWVRRHQTNNLQPRWKAPYTVLLTTPTALKVDGIAAWIHAAHVKAATTPPAGTASGPTWKVQRSQNPLKIRLTRGAP

SEQ ID NO: 54 - protein, 27 aa, Rauscher Spleen Focus-Forming Virus
YNHTSALKRECCFYADHTGLVRDSMAK

SEQ ID NO: 55 - protein, 408 aa, Rauscher Spleen Focus-Forming Virus; identical to SEQ ID NO: 52

SEQ ID NO: 56 - protein, 116 aa, Rauscher Spleen Focus-Forming Virus; identical to SEQ ID NO: 53

(19) United States
(12) Patent Application Publication
(10) Pub. No.: US 2013/0309245 A1
(43) Pub. Date: Nov. 21, 2013

(54) USING A CYTOKINE SIGNATURE TO DIAGNOSE DISEASE OR INFECTION

(71) Applicant: Whittermore Peterson Institute for Neuro-Immune Disease, Sparks, NV (US)

(72) Inventors: Vincent C. Lombardi, Reno, NV (US); Judy A. Mikovits, Reno, NV (US)

(73) Assignee: Whittermore Peterson Institute for Neuro-Immune Disease, Sparks, NV (US)

(21) Appl. No.: 13/957,932

(22) Filed: Aug. 2, 2013

Related U.S. Application Data
(63) Continuation-in-part of application No. PCT/US2012/023876, filed on Feb. 3, 2012.
(60) Provisional application No. 61/439,328, filed on Feb. 3, 2011.

(51) Int. Cl. G01N 33/68

(52) U.S. Cl. CPC G01N 33/6869; G01N 33/6863; G01N 33/6866
USPC 424/158.1; 435/5; 435/7.92; 506/18; 506/9

(57) ABSTRACT

Provided are methods and compositions for detection of levels, activity, or expression of cytokines so as to determine a cytokine signature. A cytokine signature of a subject can be compared to a control or reference value(s) and differences there between used in the diagnosis or monitoring of a neuroimmune disease or a retroviral infection.

### CROSS-REFERENCE TO RELATED APPLICATIONS

This application is a Continuation-in-Part of PCT International Application No. PCT/2012/023876 filed 3 Feb. 2012, which claims the benefit of U.S. Provisional Application Ser. No. 61/439,328, filed on Feb. 3, 2011, each of which is incorporated herein by reference in its entirety.

### STATEMENT REGARDING FEDERALLY SPONSORED RESEARCH OR DEVELOPMENT

This invention was made with government support under grant number R01 AI078234-01A2 awarded by National Institutes of Health. The government has certain rights in the invention.

### MATERIAL INCORPORATED-BY-REFERENCE

The Sequence Listing, which is a part of the present disclosure, includes a computer readable form comprising nucleotide and/or amino acid sequences of the present invention. The subject matter of the Sequence Listing is incorporated herein by reference in its entirety.

### FIELD OF THE INVENTION

The present disclosure generally relates to the differences in cytokine expression seen between healthy individuals and individuals diagnosed with a neuroimmune disease or infected with a retrovirus, including those associated with a retrovirus.

### BACKGROUND OF THE INVENTION

Cytokines are cell-to-cell signals, and include proteins, peptides and glycoproteins. Cytokines are secreted by, inter alia, glial cells in the nervous system, and many immune cells. Cytokines are generally understood to encompass interleukins, interferons (lymphokines) and chemokines. Interleukins (ILs) promote the development and differentiation of T, B and hematopoietic cells in healthy individuals. The functions of at least 35 interleukins are currently known. Interferons (IFNs, or lymphokines) are synthesized and released by lymphocytes in response to the presence of pathogens, and activate MHC and STAT signaling. Chemokines are small cytokines that can stimulate chemotaxis, and are characterized by two or four conserved cysteine residues key to the folding of the peptide. Some chemokines are produced during an immune response to recruit immune cells to the site of infection; other chemokines are homeostatic and control cell migration during tissue growth and/or maintenance.

Cytokines are recognized by cognate cell-surface receptors. Binding of a cytokine to its receptor triggers intracellular signaling which can ultimately up- or down-regulate genes and alter cell functions. The effect of any given cytokine is dependent on its identity, abundance, and the cell type on which the receptor is located.

Cytokines are immunomodulating agents, and can be proteins, peptides or glycoproteins. Cytokines are classified as interferons (lymphokines), interleukins and chemokines, based on their presumed or known function; what cells they are secreted by; or which cells they target. There is, however, much cross-classification and overlap in organization within these categories, consistent with the pleiotropic nature of the molecules' functions. Some cytokines are redundant in function with other cytokines, and pleiotropic in their activity. They can be classified into two functional types: (i) type 1 cytokines that upregulate cellular immune responses, and include IFN-γ and TGF-β among others; and (ii) type 2 cytokines which upregulate antibody responses, and include IL-4, IL-10, IL-13, among others.

Inteferons and lymphokines are secreted by lymphocytes and include, but are not limited to, interleukin (IL)-2, IL-3, IL-4, IL-5, IL-6, granulocyte-macrophage colony-stimulating factor (GM-CSF), and interferon (IFN)-γ. They recruit macrophages and other lymphocytes to sites of infection and prepare the recruited cells to mount an immune response.

Interleukins are secreted by a wide variety of cells and function to promote the development and differentiation of T, B and hematopoietic cells. Interleukins include, but are not limited to, IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35.

Chemokines are a group of smaller cytokines, initially so named because they cause chemotaxis. Some chemokines are considered pro-inflammatory, and recruit cells of the immune system to a site of infection; others are considered homeostatic and control the migration of cells during normal tissue growth and maintenance. Chemokines can be divided into four groups based on the presence and placement of up to six cysteine residues within the peptide.

The CC chemokines have two adjacent cysteines near their amino terminus. This group includes MCP-1 (or CCL2) and RANTES (or CCL5). The group also includes CCL1 (I-309, TCA-3), CCL3 (MIP-1α), CCL4 (MIP-1β), CCL6 (C10, MRP-2), CCL7 (MARC, MCP-3), CCL8 (MCP-2), CCL9 (same as CCL10; MRP-2, CCF18, MIP-113), CCL11 (Eotaxin), CCL12 (MCP-5), CCL13 (MCP-4, NCC-1, Ckβ10), CCL14 (HCC-1, MCIF, Ckβ1, NCC-2, CCL), CCL15 (Leukotactin-1, MIP-5, HCC-2, NCC-3), CCL16 (LEC, NCC-4, LMC, Ckβ12), CCL17 (TARC, dendrokine, ABCD-2), CCL18 (PARC, DC-CK1, AMAC-1, Ckβ7, MIP-4), CCL19 (ELC, Exodus-3, Ckβ11), CCL20 (LARC, Exodus-1, Ckβ4), CCL21 (SLC, 6Ckine, Exodus-2, Ckβ9, TCA-4), CCL22 (MDC, DC/β-CK), CCL23 (MPIF-1, Ckβ8, MIP-3), CCL24 (Eotaxin-2, MPIF-2, Ckβ6), CCL25 (TECK, Ckβ15), CCL26 (Eotaxin-3, MIP-4a, IMAC, TSC-1), CCL27 (CTACK, ILC, Eskine, PESKY, skinkine), and CCL28 (MEC).

In the CXC chemokines, the two amino-terminal cysteines are separated by one amino acid. These chemokines are also referred to as α-chemokines; and can be subdivided into glutamic acid-leucine-arginine (ELR) positive or negative, based on the presence or absence of this 3-aa motif before the first cysteine of the CXC motif. The CXC chemokines include, but are not limited to, CXCL1(Gro-α, GRO1, NAP-3, KC), CXCL2 (Gro-β, GRO2, MIP-2α), CXCL3 (Gro-, GRO3, MIP-2β), CXCL4 (PF-4), CXCL5 (ENA-78), CXCL6 (GCP-2), CXCL7 (NAP-2, CTAPIII, β-Ta, PEP), CXCL8 (IL-8, NAP-1, MDNCF, GCP-1), CXCL9 (MIG, CRG-10), CXCL10 (IP-10, CRG-2), CXCL11 (I-TAC, β-R1, IP-9), CXCL12 (SDF-1, PBSF), CXCL13 (BCA-1, BLC), CXCL14 (BRAK, bolekine), CXCL15 (Lungkine, WECHE), CXCL16 (SRPSOX), and CXCL17 (DMC, VCC-10).

The C chemokines (or γ chemokines) have only two cysteines, one of which is near the N-terminus of the peptide, and one of which is near the C-terminus. The two C chemokines are XCL1 (lymphotactin-α, SCM-1a, ATAC) and XCL2 (lymphotactin-β, SCM-1β). The CX₃C chemokine CX3CL2 (Fractalkine, Neurotactin, ABCD-3) has three amino acids between the two N-terminal cysteine residues.

The cytokines RANTES, MIP (macrophage inflammatory proteins) 1α and 1β (now known as CCL5, CCL3 and CCL4 respectively) suppress HIV-1 (Ciccgu et al., Science 270(5243): 1811-1815, 1995). It has been suggested that increased amounts of these chemokines is associated with more favorable clinical status in AIDS cases (Garzino-Demo et al., PNAS 96(21):11986-11991, 1999).

It has been reported that initial HIV infection disrupts the normal balance of cytokines by causing the levels of certain cytokines to rise. Cytokines reported to increase during initial HIV infection include IFNγ, IL-2 and IL-12. As HIV progresses to AIDS, the steady-state levels of IFNγ, IL-2 and IL-12 are reported to fall. Simultaneously, the levels of another group of cytokines (including IL-4, IL-5, IL-6, IL-10, TNFα) have been reported to increase. According to the Th-1/Th-2 theory, this change in cytokine expression signature may directly cause many of the symptoms associated with AIDS (Babakhanian, 1995).

Neuroimmune disease is a category of diseases which have both neurological effects and (auto)immune effects. Neuroimmune diseases can be chronic neuroimmune diseases, or acute neuroimmune diseases. As used herein, neuroimmune disease can include chronic fatigue syndrome, fibromyalgia, myalgic encephalitis, atypical multiple sclerosis, non-epileptic seizures, Gulf War Syndrome or autism.

Chronic Fatigue Syndrome (CFS) is an example of a neurological disease believed to involve malfunctions in the immune system. CFS is a debilitating disease that affects more than one million people in the US alone. CFS is a disease characterized by severe and debilitating fatigue, sleep abnormalities, impaired memory and concentration, and musculoskeletal pain. In the Western world, the population prevalence is estimated to be of the order of 0.5%-2% (Papanicolaou et al. 2004. Neuroimmunomodulation 11(2):65-74; White. 2007. Popul Health Metr 5(1):6). CFS subjects are known to have a shortened lifespan and are at risk for developing lymphoma. Currently, there is no diagnostic test and no treatment, except for the specific treatment of microbial infections in those cases in which microbial agents can be identified (Devanur and Kerr. 2006. J Clin Virol 37(3):139-150). Although the precise pathogenesis of CFS is unknown, a range of factors have been shown to contribute (Komaroff and Buchwald. 1998. Annu Rev Med 49:1-13; Devanur and Kerr. 2006. supra). Furthermore, a single patient with a bona fide CFS diagnosis can present with variable symptoms over the duration of the illness.

Several retroviruses such as the MuLVs, primate retroviruses, HIV, HTLV-1 and xenotropic murine leukemia virus-related virus (XMRV) are associated with neurological diseases (C. Power, Trends in Neurosci. 24, 162, 2001; Miller and Meucii 1999 TINS 22(10), 471-479; Power et al. 1994 Journal of Virology 68(7) 4463-4649). Investigation of the molecular mechanism of retroviral induced neurodegeneration in rodent models revealed vascular and inflammatory changes mediated by cytokines and chemokines and these changes were observed prior to any neurological pathology (X. Li, C., Hanson. J. Cmarik, S. Ruscetti J. Virol. 83, 4912, March, 2009, K. E. Peterson., B Chesebro. Curr. Opin. Microbiol. Immunol. 303, 67 2006). The XMRV genome encodes, in 5′-to-3′ order, the 5′ long terminal repeat (LTR); a short, apparently non-coding sequence comprising a splice site acceptor (“SA”); the Gag gene; the Pro-Pol gene, comprising a splice donor site (“SD”), the extreme 3′-end of which overlaps with the 5′-end of the Env gene; the Env gene; another short non-coding sequence; the 3′-end LTR; and a poly-A tail (see Urisman et al. 2006 PLoS Pathogens 2(3), e25; Lombardi et al. 2009 Science 326(5952), 585-589).

### SUMMARY OF THE INVENTION

Among the various aspects of the present invention is the provision of a method of predicting symptoms in a subject infected with a retrovirus.

One aspect provides a method of diagnosing a retroviral infection or a neuroimmune disease in a subject. In some embodiments, the method includes comparing a cytokine expression signature of a subject with a control. In some embodiments, the cytokine expression signature includes an expression level of at least three cytokines or chemokines, which can be selected from IL-8, IL-13, MIP-1β, TNF-α, MCP-1, IL-7, IFN-α, IL-6, MIP-1α, or GM-CSF. In some embodiments, the method includes diagnosing the subject with a retroviral infection or a neuroimmune disease where the cytokine expression signature of the subject comprises at least one of the selected cytokines or chemokines at or above a predetermined threshold of expression.

In some embodiments, the method includes application of an algorithm for determining whether a cytokine signature is indicative of a retroviral infection or a neuroimmune disease in a subject. In some embodiments, the algorithm includes a weighted value for a portion of or all of cytokines or chemokines of the cytokine signature. In some embodiments, the algorithm includes addition of a weighted value to arrive at a sum of weighted values where a cytokine or chemokine of the cytokine expression signature is at or above a predetermined threshold of expression. In some embodiments, application of an algorithm includes diagnosing the subject with a retroviral infection or a neuroimmune disease where the sum of weighted values is at or above a predetermined threshold value.

Another aspect provides a device for detecting a cytokine expression signature of a subject comprising an array, wherein the array detects the presence or expression level at least three cytokines or chemokines selected from the group consisting of IL-8, IL-13, MIP-1β, TNF-α, MCP-1, IL-7, IFN-α, IL-6, MIP-1α, and GM-CSF.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1 shows the importance of various cytokines and chemokines in XMRV-related disease as assessed by Random Forests Variables analysis.

FIG. 2 shows the results of cluster analysis of cytokine/chemokine expression data in XMRV-infected subjects, XMRV-infected subjects with increased γδ T-cell populations, and healthy controls.

FIG. 3 shows the results of Random Forests variable analysis for subject 2623.

FIG. 4 shows the results of Random Forests variable analysis for subject 1127.

FIG. 5 shows the results of Random Forests variable analysis for subject 967.

### DETAILED DESCRIPTION OF THE INVENTION

Provided herein is a description of signature changes in cytokine expression that can be reliably associated with a diagnosis of a neuroimmune disease, such as CFS, or with a retroviral infection. The present disclosure is based, at least in part, on the observation that cytokine expression in an individual diagnosed with chronic fatigue syndrome (CFS) is different from cytokine expression in a healthy individual. The present disclosure is based, at least in part, on the correlation of specific changes in cytokine expression with a diagnosis of CFS. The present disclosure is also based, at least in part, on the correlation of specific changes in cytokine expression with a retroviral infection.

The inventors have identified a statistically significant dysregulation in the innate immune system in a population of CFS patients when compared to healthy controls. Specifically, it has been observed that, i) plasma levels of interferon alpha (IFN-α) are significantly decreased in CFS patients (p<0.0001), ii) IL-8, IL-6, TNF-α, MIP-1α, MIP-1β, IP-10, and MCP-1 are significantly upregulated in this population; and iii) plasmacytoid dentritic cells (pDCs), when isolated from CFS patients and subjected to the Toll-like receptor (TLR) 7 agonists imiquimod and to a lesser extent, the TLR9 agonist ODN 2213, overproduce the pro-inflammatory cytokines IL-6, TNF-α, MIP-1α, MIP-1β, IP-10, MCP-1, and IFN-α in contrast to pDCs isolated from healthy controls. When taken together, these data implicate the involvement of a dysregulation of plasmacytoid dentritic cells in the pathophysiology of CFS.

Cytokine Signature

A cytokine expression signature of a subject, as described herein, can include changes in level, activity, or expression of one or more cytokines for which no or substantially no corresponding changes occur in a control. For example, a cytokine expression signature of a subject can include changes in level, activity, or expression of one or more of a type 1 cytokine or a type 2 cytokine.

A cytokine expression signature of a subject can include changes in level, activity, or expression of one or more of an inteferon or a lymphokine. For example, a cytokine expression signature of a subject can include changes in level, activity, or expression of one or more of interleukin (IL)-2, IL-3, IL-4, IL-5, IL-6, granulocyte-macrophage colony-stimulating factor (GM-CSF), and interferon (IFN)-γ. As another example, a cytokine expression signature of a subject can include changes in level, activity, or expression of one or more of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, and IL-35.

A cytokine expression signature of a subject can include changes in level, activity, or expression of one or more chemokines. For example, a cytokine expression signature of a subject can include changes in level, activity, or expression of one or more of a CC chemokine, a CXC chemokine, a C chemokine (or γ chemokine), RANTES, CCL5, CCL3 and CCL4.

For example, a cytokine expression signature of a subject can include changes in level, activity, or expression of one or more of CC chemokines selected from MCP-1 (or CCL2), RANTES (or CCL5), CCL1 (I-309, TCA-3), CCL3 (MIP-1α), CCL4 (MIP-1β), CCL6 (C10, MRP-2), CCL7 (MARC, MCP-3), CCL8 (MCP-2), CCL9 (same as CCL10; MRP-2, CCF18), CCL11 (Eotaxin), CCL12 (MCP-5), CCL13 (MCP-4, NCC-1, Ckβ10), CCL14 (HCC-1, MCIF, Ckβ1, NCC-2, CCL), CCL15 (Leukotactin-1, MIP-5, HCC-2, NCC-3), CCL16 (LEC, NCC-4, LMC, Ckβ12), CCL17 (TARC, dendrokine, ABCD-2), CCL18 (PARC, DC-CK1, AMAC-1, Ckβ7, MIP-4), CCL19 (ELC, Exodus-3, Ckβ11), CCL20 (LARC, Exodus-1, Ckβ4), CCL21 (SLC, 6Ckine, Exodus-2, Ckβ9, TCA-4), CCL22 (MDC, DC/β-CK), CCL23 (MPIF-1, Ckβ8, MIP-3), CCL24 (Eotaxin-2, MPIF-2, Ckβ6), CCL25 (TECK, Ckβ15), CCL26 (Eotaxin-3, MIP-4a, IMAC, TSC-1), CCL27 (CTACK, ILC, Eskine, PESKY, skinkine), and CCL28 (MEC).

As another example, a cytokine expression signature of a subject can include changes in level, activity, or expression of one or more of CXC chemokines selected from a glutamic acid-leucine-arginine (ELR) positive CXC chemokine or a glutamic acid-leucine-arginine (ELR) negative CXC chemokine. As another example, a cytokine expression signature of a subject can include changes in level, activity, or expression of one or more of a CXC chemokine selected from CXCL1(Gro-α, GRO1, NAP-3, KC), CXCL2 (Gro-β, GRO2, MIP-2α), CXCL3 (Gro-, GRO3, MIP-2β), CXCL4 (PF-4), CXCL5 (ENA-78), CXCL6 (GCP-2), CXCL7 (NAP-2, CTAPIII, β-Ta, PEP), CXCL8 (IL-8, NAP-1, MDNCF, GCP-1), CXCL9 (MIG, CRG-10), CXCL10 (IP-10, CRG-2), CXCL11 (I-TAC, β-R1, IP-9), CXCL12 (SDF-1, PBSF), CXCL13 (BCA-1, BLC), CXCL14 (BRAK, bolekine), CXCL15 (Lungkine, WECHE), CXCL16 (SRPSOX), and CXCL17 (DMC, VCC-10).

As another example, a cytokine expression signature of a subject can include changes in level, activity, or expression of one or more C chemokines selected from XCL1 (lymphotactin-α, SCM-1α, ATAC), XCL2 (lymphotactin-β, SCM-1β), and CX3CL2 (Fractalkine, Neurotactin, ABCD-3).

As another example, a cytokine expression signature of a subject can include changes in level, activity, or expression of one or more RANTES, CCL5, CCL3, and CCL4.

As another example, a cytokine expression signature of a subject can include one or more of those cytokines known to be upregulated by pDCs (e.g., IL-8, IL-6, TNF-α, MIP-α or MIP-1β). As another example, a cytokine expression signature of a subject can exclude one or more of those cytokines not known to be upregulated by pDCs (e.g., IL-1a, IL-2, IL-3, IL-4, IL-5, IL-13 or IL-15).

It is understood that a cytokine expression signature, as described herein, can include any combinations of cytokines recited above for which there is a change in expression in a subject as compared to a control. Particular combinations are further discussed below.

A cytokine expression signature as described herein can include an expression pattern in which one or more cytokines are modulated in a subject as compared to a control. For example, a cytokine expression signature can include an expression pattern in which one or more cytokines are upregulated in a subject as compared to a control. As another example, a cytokine expression signature can include an expression pattern in which one or more cytokines are down regulated in a subject as compared to a control. As another example, a cytokine expression signature can include a cytokine expression pattern in which one or more cytokines are upregulated and one or more other cytokines are down regulated in a subject as compared to a control.

A cytokine expression signature can include any combination of increase(s) and decrease(s) in the expression levels of any of the cytokines described herein. A cytokine that has an altered expression can include any cytokine identified herein. Alteration in cytokine expression can include both up- or down-regulation of expression. Such alterations can be part of a cytokine expression signature as described herein.

Upregulated

A cytokine expression signature can include expression of at least one cytokine upregulated in a subject as compared to a control. For example, a cytokine expression signature can include at least two, at least three, at least four, at least five, at least six, at least seven, at least eight, at least nine, or at least ten or more cytokines upregulated in a subject as compared to a control.

A cytokine (of a cytokine signature) that has a upregulated expression can be selected from IL-8, MIP-1β, TNF-α, IL-6, IL-2, IP-10, Eotaxin, IL-12, Regulated on Activation, Normal T Expressed and Secreted protein (RANTES), MCP-1 and MIP-1α. A cytokine signature of a subject can comprise upregulated expression of one or more of cytokines selected from IL-8, MIP-1β, TNF-α, IL-6, IL-2, IP-10, Eotaxin, IL-12, Regulated on Activation, Normal T Expressed and Secreted protein (RANTES), MCP-1 and MIP-1α.

A cytokine signature can include IL-8 expression at least about 10-fold higher in a subject as compared to a control. For example, IL-8 expression can be at least about 20-, at least about 30-, at least about 40-, at least about 50-, at least about 60-, at least about 70-, at least about 80-, or at least about 90-fold higher in a subject as compared to a control. As another example, IL-8 expression can be at least about 100-fold or more higher in a subject as compared to a control.

A cytokine signature can include MIP-1β expression at least about 10-fold higher in a subject as compared to a control. For example, MIP-1β expression can be at least about 20-, at least about 30-, at least about 40-, at least about 50-, at least about 60-, at least about 70-, at least about 80-, or at least about 90-fold higher in a subject as compared to a control. As another example, MIP-1β expression can be at least about 100-fold or more higher in a subject as compared to a control.

A cytokine signature can include TNF-α expression at least about 2-fold higher in a subject as compared to a control. For example, TNF-α expression can be at least about 3-, at least about 4-, at least about 5-, at least about 6-, at least about 7-, at least about 8-, or at least about 9-fold higher in a subject as compared to a control. As another example, TNF-α expression can be at least about 10-fold or more higher in a subject as compared to a control.

A cytokine signature can include IL-6 expression at least about 2-fold higher in a subject as compared to a control. For example, IL-6 expression can be at least about 3-, at least about 4-, at least about 5-, at least about 6-, at least about 7-, at least about 8-, or at least about 9-fold higher in a subject as compared to a control. As another example, IL-6 expression can be at least about 10-fold or more higher in a subject as compared to a control.

A cytokine signature can include IL-2 expression at least about 2-fold higher in a subject as compared to a control. For example, IL-2 expression can be at least about 3- or at least about 4-fold higher in a subject as compared to a control. As another example, IL-2 expression can be at least about 5-fold or more higher in a subject as compared to a control.

A cytokine signature can include IP-10 expression at least about 2-fold higher in a subject as compared to a control. For example, IP-10 expression can be at least about 3-fold higher in a subject as compared to a control. As another example, IP-10 expression can be at least about 4-fold or more higher in a subject as compared to a control.

A cytokine signature can include Eotaxin expression at least about 2-fold higher in a subject as compared to a control. For example, Eotaxin expression can be at least about 3-fold higher in a subject as compared to a control. As another example, Eotaxin expression can be at least about 4-fold or more higher in a subject as compared to a control.

A cytokine signature can include IL-12 expression at least about 1.1-fold higher in a subject as compared to a control. For example, IL-12 expression can be at least about 1.2-fold or more higher in a subject as compared to a control.

A cytokine signature can include RANTES expression at least about 2-fold higher in a subject as compared to a control. For example, RANTES expression can be at least about 3-fold higher in a subject as compared to a control. As another example, RANTES expression can be at least about 4-fold or more higher in a subject as compared to a control.

A cytokine signature can include MCP-1 expression at least about 1.1-fold higher in a subject as compared to a control. For example, MCP-1 expression can be at least about 1.2-fold or more higher in a subject as compared to a control.

A cytokine signature can include MIP-1α expression at least about 2-fold higher in a subject as compared to a control. For example, MIP-1α expression can be at least about 3-, at least about 4-, at least about 5-, at least about 6-, at least about 7-fold or more higher in a subject as compared to a control.

Down Regulated

A cytokine expression signature can include expression of at least one cytokine down regulated in a subject as compared to a control. For example, a cytokine expression signature can include at least two, at least three, at least four, at least five, at least six, at least seven, at least eight, at least nine, or at least ten or more cytokines down regulated in a subject as compared to a control.

A cytokine (of a cytokine signature) that has down regulated expression can be selected from IL-13, IL-5, IL-7, MIG, and IFN-α. A cytokine signature of a subject can comprise down regulated expression of one or more of cytokines selected from IL-13, IL-5, IL-7, MIG, and IFN-α.

A cytokine signature can include IL-13 expression at least about 2-fold lower in a subject as compared to a control. For example, IL-13 expression can be at least about 3-, at least about 4-, or at least about 5-fold or more lower in a subject as compared to a control.

A cytokine signature can include IL-5 expression can be at least about 2-fold lower in a subject as compared to a control. For example, IL-5 expression can be at least about 3- or at least about 4-fold or more lower in a subject as compared to a control.

A cytokine signature can include IL-7 expression at least about 2-fold lower in a subject as compared to a control. IL-7 expression can be at least about 3-, at least about 4-, or at least about 5-fold or more lower in a subject as compared to a control.

A cytokine signature can include MIG expression can be at least about 2-fold lower in a subject as compared to a control.

A cytokine signature can include IFN-α expression at least about 2-fold lower in a subject as compared to a control.

A cytokine signature can include GM-CSF expression at least about 0.7-fold lower in a subject as compared to a control.

Combinations

A cytokine expression signature can include the changes in a cytokine expression described herein. For example, a cytokine expression signature can include changes in level, activity, or expression of one or more cytokines selected from GM-CSF, IL-8, MIP-1β, TNF-α, IL-6, IL-2, IP-10, Eotaxin, IL-12, Regulated on Activation, Normal T Expressed and Secreted protein (RANTES), MCP-1, MIP-1α, IL-13, IL-5, IL-7, MIG, and IFN-α, as compared to a control.

As another example, a cytokine expression signature can include changes in level, activity, or expression of IL-8, IL-13, MIP-1β, TNF-α, MCP-1, IL-7, IFN-α, IL-6, MIP-1α, and GM-CSF, as compared to a control.

A cytokine expression signature of a subject can include changes in level, activity, or expression of two or more of: (i) IL-8 expression of at least about 10-fold higher in a subject, as compared to a control; (ii) IL-13 expression of at least about 5-fold lower in a subject, as compared to a control; (iii) MIP-1β expression of at least about 10-fold higher in a subject, as compared to a control; (iv) TNF-α expression of at least about 10- or more-fold higher in a subject, as compared to a control; (v) MCP-1 expression of at least about 1.1-fold higher in a subject, as compared to a control; (vi) IL-7 expression of at least about 5-fold lower in a subject, as compared to a control; (vii) IFN-α expression of at least about 2-fold lower in a subject, as compared to a control; (viii) IL-6 expression of at least about 10- or more-fold higher in a subject, as compared to a control; (ix) MIP-1α expression of at least about 2-fold higher in a subject, as compared to a control and (x) GM-CSF expression of at least about 0.7-fold higher in a subject, as compared to a control. For example, a cytokine expression signature of a subject can include changes in level, activity, or expression of three or more of (i)-(x). As another example, a cytokine expression signature of a subject can include changes in level, activity, or expression of four or more of (i)-(x). As another example, a cytokine expression signature of a subject can include changes in level, activity, or expression of five or more of (i)-(x). As another example, a cytokine expression signature of a subject can include changes in level, activity, or expression of six or more of (i)-(x). As another example, a cytokine expression signature of a subject can include changes in level, activity, or expression of seven or more of (i)-(x). As another example, a cytokine expression signature of a subject can include changes in level, activity, or expression of eight or more of (i)-(x). As another example, a cytokine expression signature of a subject can include changes in level, activity, or expression of nine or more of (i)-(x). As another example, a cytokine expression signature of a subject can include changes in level, activity, or expression of (i)-(x). The magnitude of change of the level, activity, or expression of cytokines above are exemplary and can include any of the level of change described herein for a particular cytokine.

Cytokine Detection

Detection or determination of a cytokine, cytokine activity, expression of a cytokine, or expression levels of a cytokine can be according to conventional methods understood in the art (see e.g., Kotb and Calandra 2010 Cytokines and Chemokines in Infectious Diseases Handbook, Humana Press, 1^{st} ed., ISBN-10 1617372471; Kuchroo et al. 2011 Cytokines and Autoimmune Diseases, Humana Press, 1^{st} ed., ISBN-10 1617372250; House and Descotes 2010 Cytokines in Human Health: Immunotoxicology, Pathology, and Therapeutic Applications, Methods in Pharmacology and Toxicology, Humana Press, 1^{st} ed., ISBN-10 1617375861; DeLey 2010 Cytokine Protocols, Methods in Molecular Biology, Humana Press, 1^{st} ed., ISBN-10 1617372692; Korholz and Kiess 2010 Cytokines and Colony Stimulating Factors, Methods and Protocols, Methods in Molecular Biology, Humana Press, 1^{st} ed., ISBN-10 1617373184). For example, identification of a cytokine can be according to a cell secretion assay (see e.g., Manz et al. 1995 PNAS 92, 1921-1925). As another example, identification of a cytokine can be according to assays described herein.

Prediction Algorithm

In some embodiments, a predictive algorithm can provide weighting for presence or magnitude of level, activity, or expression of different combinations of cytokines. An individual cytokine can be assigned a value of relative importance. When that cytokine is present at or above a threshold level (e.g., as described above), the value of relative importance for that cytokine can be added to a total value representing the cytokine signature. Where the total value exceeds a threshold, then a prediction or diagnosis of a neuroimmune disorder (e.g., CFS) or retroviral infection can be made (see e.g., Example 5).

For example, an individual cytokine at or above a threshold level can be assigned a weighted value such as: IL-8 is 100, IL-13 is 90, MIP-1β is 80, TNF-α is 70, MCP-1 is 60, IL-7 is 50, IFN-α is 40, IL-6 is 30, MIP-1α is 20, and GM-CSF is 10. A prediction or diagnosis of a neuroimmune disorder (e.g., CFS) or retroviral infection can be made by any combination of cytokines with a combined value of about 190 or greater, about 200 or greater, about 210 or greater, about 220 or greater, about 230 or greater, about 240 or greater, about 250 or greater, or more.

For example, an individual cytokine at or above a threshold level can be assigned a weighted value such that IL-8 is 100, IL-13 is 90, MIP-1β is 80, TNF-α is 70, MCP-1 is 60, IL-7 is 50, IFN-α is 40, IL-6 is 30, MIP-1α is 20, and GM-CSF is 10, and a prediction or diagnosis of a neuroimmune disorder (e.g., CFS) or retroviral infection can be made by any combination of cytokines or chemokines with a combined value of about 210 or greater.

Correlation of Cytokine Expression Signature with Neuroimmune Disease

The present inventors have determined that a cytokine expression signature, as described herein, can be correlated with a diagnosis of a neuroimmune disease. For example, a cytokine expression signature can be correlated with a diagnosis of neuroimmune disease associated with a retroviral infection. As another example, a cytokine expression signature can be correlated with a diagnosis of neuroimmune disease not presently known to be associated with a retroviral infection.

A cytokine expression signature associated with a neuroimmune disease can include an expression pattern in which one or more cytokines are modulated (e.g., upregulated or down regulated) in a subject having or diagnosed as having the neuroimmune disease.

A neuroimmune disease that is correlated with a cytokine expression signature can be a chronic neuroimmune disease. A neuroimmune disease correlated with a cytokine expression signature can be, for example, chronic fatigue syndrome, fibromyalgia, myalgic encephalitis, atypical multiple sclerosis, non-epileptic seizures, Gulf War Syndrome or autism.

A cytokine expression signature associated with a neuroimmune disease can include an expression level of any combination of cytokines as described herein. A cytokine of an expression signature and level, activity, or expression relative to a control can be as discussed herein.

A cytokine expression signature associated with a neuroimmune disease can be according to any of the cytokine expression signatures discussed herein. For example, a cytokine expression signature can include changes in level, activity, or expression of one or more cytokines selected from GM-CSF, IL-8, MIP-1β, TNF-α, IL-6, IL-2, IP-10, Eotaxin, IL-12, Regulated on Activation, Normal T Expressed and Secreted protein (RANTES), MCP-1, MIP-1α, IL-13, IL-5, IL-7, MIG, and IFN-α. As another example, a cytokine expression signature associated with a neuroimmune disease can include changes in level, activity, or expression of IL-8, IL-13, MIP-1β, TNF-α, MCP-1, IL-7, IFN-α, IL-6, MIP-1α, and GM-CSF. Presence or magnitude of upregulated or down regulated level, activity, or expression of a cytokine can be according to the discussion above.

A control for the purposes of a cytokine expression signature associated with a neuroimmune disease can be, for example, the expression signature of the same or similar group of cytokines in a subject not having or diagnosed as having the neuroimmune disease. As another example, a control for the purposes of a cytokine expression signature associated with a neuroimmune disease can be reference levels of the same or similar group of cytokines. As another example, a control for the purposes of a cytokine expression signature associated with a neuroimmune disease can be expression levels of the same or similar group of cytokines in the same subject at a point in time in which that subject was healthy or did not have or was not diagnosed as having the neuroimmune disease.

Correlation of Cytokine Expression Signature with a Retroviral Infection

The present inventors have discovered that a retroviral infection can be correlated with alterations in cytokine expression. A cytokine expression signature associated with a retroviral infection can include an expression pattern in which one or more cytokines are modulated (e.g., upregulated or down regulated) in a subject infected with a retrovirus relative to a subject who is not infected with the retrovirus.

A retrovirus as that term is used herein can be, for example, a gamma retrovirus.

A retrovirus as that term is used herein can be, for example, a MuLVs, primate retrovirus, HIV, HTLV-1 or xenotropic murine leukemia virus-related virus (XMRV) (see Power, Trends in Neurosci. 24, 162, 2001; Miller and Meucii 1999 TINS 22(10), 471-479; Power et al. 1994 Journal of Virology 68(7) 4463-4649)).

A retrovirus as that term is used herein can be, for example, a retrovirus as described in U.S. Pat. App. Pub. No. 2011/0311484, filed Apr. 6, 2011, incorporated herein by reference in its entirety. A retrovirus as that term is used herein can have a gamma retroviral associated function or activity and be encoded by a sequence at least about 80% sequence identity (e.g., at least about 85%, at least about 90%, at least about 91%, at least about 92%, at least about 93%, at least about 94%, at least about 95%, at least about 96%, at least about 97%, at least about 98%, or at least about 99% sequence identity) to a sequence according to SEQ ID NO: 1 and, optionally, having one or more nucleotide changes selected from C80T, G90A, A96G, A97G, G111A, A137-157 deletion, T173C, G180A, G183A, C197T, C247T, C257T, C308T, C308G, C319T, C320T, T326C, A329G, C715T, T791G, A804G, T816Del, A856G, A665Del, T691G, G790A (potential hypermethylation site), T791G, T796C, G807Del, A840G, A873G, A875G, C903T, T963G, C5810Del, A6101T, G6154T, G7421A, A7459C, and an insertion at nucleotide position 7322 having a sequence of GAAAAGTCTCTGACCTCGTTGTCTGAGGTGGTCCTACAGAACCGGAGGGGAT TAGTCTA (SEQ ID NO: 179); or a functional fragment thereof. For example, an XMRV strain can have at least two, at least three, at least four, at least five, at least six, at least seven, at least eight, at least nine, or at least ten, or more, of nucleotide changes described herein. Assays for determining gamma retrovirus functionality can be according to general methods known in the art (see e.g., Kurth 2010 Retroviruses: Molecular Biology, Genomics and Pathogenesis, Caister Academic Press, ISBN-10: 1904455557; Zhu 2010 Human Retrovirus Protocols: Virology and Molecular Biology (Methods in Molecular Biology), 1st Edition, Humana Press, ISBN-10: 1617375993) and those described in U.S. Pat. App. Pub. No. 2011/0311484.

A retrovirus as that term is used herein can be, for example, a xenotropic murine leukemia virus-related virus (XMRV). An XMRV can be according to a virus described in, for example, Urisman et al. 2006 PLoS Pathogens 2(3), e25; Lombardi et al. 2009 Science 326(5952), 585-589; Silverman et al. WO2006110589; Mikovits et al. US App Pub No. 2010/0167268; Mikovits et al. WO2010/148323; Mikovits at al. US App Pub. No. 2011/0117056; and Mikovits et al. US App Pub. No. 2011/0151431, each of which are incorporated herein by reference in their entirety. The XMRV consensus sequence has been described previously (Urisman et al., PLOS Pathogens 2006 2(3):e25), Accession number DQ399707.1, and is referred to herein as VP62, or SEQ ID NO: 1. VP62 was identified from a clone reconstructed from nucleic acids isolated from prostate tumors. Accession number EF185282.1 (SEQ ID NO: 162) is an 8165 nucleotide sequence of VP62, while Accession number DQ399707.1 (SEQ ID NO: 1) is an 8185 nucleotide sequence of VP62. The reference sequence of SEQ ID NO: 1 corresponds to Accession number DQ399707.1.

A number of clinical observations, previously described in CFS, suggest a defect in the innate immune response. For instance, viral agents such as parvovirus B19, cytomegalovirus (CMV), Epstein-Barr virus (EBV) and human herpes virus 6 and 7 (HHV-6 and 7), have been associated with CFS (reviewed by Devanur et al. J Clin Virol, 2006. 37(3): p. 139-50). Most individuals encounter these viruses early in life; however, they are kept in check by the immune system and only reactivate at times of immune suppression. Therefore, the viral reactivation frequently observed in CFS patients suggests suppression of the antiviral immune system. A number of antiviral mechanisms depend on the regulation of type I IFN for proper function. For example, the 2′-5′ oligoadenylate synthetase enzymes (OAS), the endoribonuclease L (RNase L) and protein kinase R (PKR) are regulated by type I IFN (Stark et al., Annu Rev Biochem, 1998. 67: p. 227-64); and this pathway has been reported to be dysregulated in CFS patients (see e.g., De Meirleir et al., Am J Med, 2000. 108(2): p. 99-105; De Meirleir et al., Clin Infect Dis, 2002. 34(10): p. 1420-1; author reply 1421-2; Fremont et al., Life Sci, 2006. 78(16): p. 1845-56). A dysregulation in the type I IFN response is consistent with the viral reactivation observed in CFS. Another salient clinical observation consistently described in CFS is the unregulated overproduction of pro-inflammatory cytokines, such as IL-8, IL-6 and TNF-α (Fletcher et al., J Transl Med, 2009. 7: p. 96; Kerr and Tyrell, Curr Pain Headache Rep, 2003. 7(5): p. 333-41; Lombardi et al., In Vivo, 2011. 25(2); Peterson et al., Clin Diagn Lab Immunol, 1994. 1(2): p. 222-6). The over expression of these cytokines may be responsible for many of the symptoms associated with CFS.

A cytokine expression signature that is associated with infection with a retroviral infection can include an expression level of a cytokine as described herein. A cytokine of an expression signature and expression levels relative to a control can be as discussed above.

A cytokine expression signature correlated with a retroviral infection in a subject can be according to any of the cytokine expression signatures discussed above. For example, a cytokine expression signature can include changes in level, activity, or expression of one or more cytokines selected from GM-CSF, IL-8, MIP-1β, TNF-α, IL-6, IL-2, IP-10, Eotaxin, IL-12, Regulated on Activation, Normal T Expressed and Secreted protein (RANTES), MCP-1, MIP-1α, IL-13, IL-5, IL-7, MIG, and IFN-α. As another example, a cytokine expression signature associated with infection with XMRV can include changes in level, activity, or expression of IL-8, IL-13, MIP-1β, TNF-α, MCP-1, IL-7, IFN-α, IL-6, MIP-1α, and GM-CSF. Levels of upregulated or down regulated expression for any of these cytokines can be according to the discussion above.

A control for the purposes of a cytokine expression signature associated with a retroviral infection can be, for example, the expression signature of the same or similar group of cytokines in a subject not infected with the retrovirus. As another example, a control for the purposes of a cytokine expression signature associated with a retroviral infection can be reference levels of the same or similar group of cytokines. As another example, a control for the purposes of a cytokine expression signature associated with a retroviral infection can be expression levels of the same or similar group of cytokines in the same subject at a point in time in which that subject was healthy or was not infected with the retrovirus.

An amount of retrovirus present in a subject can be associated with a degree of change of one or more cytokines of a cytokine signature. For example, an amount of a retrovirus present in a subject can be correlated to an index number describing the modulated cytokine signature. An amount of a retrovirus present in a subject can be determined according to methods known in the art, such as determination of viral titre. For example, an retrovirus viral titre of a subject or a sample of a subject can be associated with a degree of change in a cytokine signature, or one or more components thereof, of the subject or the sample of the subject. For example, increasing viral titre can be associated with an increasing change in cytokine expression from a subject who is negative for a retrovirus. As another example, increasing viral titre can correlate to a change in a cytokine expression; so that, at a relatively low retrovirus titre, a first cytokine expression signature is observed; and at a relatively higher retrovirus titre, a second cytokine expression signature is observed.

Mechanism

While under no obligation to do so, and without limiting the present invention in any way, mechanisms underlying a correlation of altered cytokine expression with retroviral infection or symptoms of neuroimmune disease are provided herein.

Single stranded RNA and CpG DNA initiate the synthesis of type I IFN through the activation TLR 7/8 and 9 respectively, where TNF receptor associated factor 6 (TRAF6) plays a pivotal role in the activation of pro-inflammatory cytokine production. A TRAF6 initiated cascade leads to phosphorylation and nuclear translocation of IRF7 and 8, consequently, triggering transcription of multiple pro-inflammatory cytokines and IFN-α. To prevent over-expression of these cytokines, Fas-associated Death Domain (FADD) interacts with the tripartite motif-containing protein 21 (TRIM21) promoting TRIM21 ubiquitin ligase activity and subsequently down-regulating cytokine production. Thus, TRIMM21 provides a negative feedback loop to prevent over-production of inflammatory cytokines. Findings described herein support that dysregulation of TRIM21 can lead to the over production of pro-inflammatory cytokines and the hyper-reactivity of IFN-α expression in CFS patients.

Viral reactivation is a common occurrence in CFS; but a mechanism to account for this condition has not been reported. As pDCs are thought to be primarily involved in responses to viral infection, the inventors propose that a pDC dysregulation may be a contributing factor to viral reactivation. Plasmacytoid dendritic cells are the primary producers of IFN-α and also produce pro-inflammatory cytokines that are consistent with previous observations in CFS. Observations reported herein are consistent with a dysregulation in the negative feedback loop for IFN-α control. CFS patients display a number of immune abnormalities, mostly involving the innate immune system; but some employ the humorial immune system as well. Plasmacytoid dendritic cells are professional antigen-presenting cells but they also produce cytokines, which activate T-cells, B-cells and NK cells. Therefore, pDCs link innate and adaptive immunity, which is a requisite to explain the pathology of CFS.

Furthermore, an interrelated dysregulation may occur in the pathways mediating type I IFN and pro-inflammatory cytokine production in pDCs of CFS. Dysregulation of pDCs may account for the aberrant IFN and pro-inflammatory cytokine production as well as the other abnormalities observed in the innate immune system of CFS patients. As shown herein, CFS patients have decreased plasma levels of INF-a. Because pDC are major producers of INF-a, it is expected that pathogenesis of CFS may be explained by dysfunction of these cells. Indeed, data demonstrated that while producing limited amount of INF-a in vivo, pDC from CFS are releasing 20 folds more INF-a when stimulated with TLR ligands in vitro as compared to healthy donors. Although the pattern of pro-inflammatory cytokine produced by stimulated pDC was similar between patients and controls, actual production was 3-20 folds higher in the CFS patients. This dysregulation is also consistent with other chronic immune diseases such as Sjogren's syndrome and systemic lupus erythematosus.

Recently, a novel intracellular antiviral function has been reported for TRIM21, involving intracellular antibody-mediated proteolysis (Mallery et al., Proc Natl Acad Sci USA. 107(46): p. 19985-90). A dysregulation of the biochemical pathway involving TRIM21, FADD or TRAF6 in pDCs suggests the origin of inflammatory cytokines in addition to the dysregulation of IFN. Plasmacytoid dendritic cells are found primarily in the gut, the spleen and the lymph nodes (Dzionek et al., Hum Immunol, 2002. 63(12): p. 1133-48). Thus the inventors propose that pDC involvement of CFS is consistent with the lymphadenopathy, splenomegaly and gastrointestinal abnormalities commonly reported in CFS patients (see Carruthers et al., Journal of Chronic Fatigue Syndrome, 2003. 11(1): p. 7115).

The tripartite motif (TRIM) family member, TRIM21, is an E3 ubiquitin ligase that is known to ubiquitinate the IFN regulatory factors IRF3, IRF7 and IRF8 through a cooperative interaction with the Fas-associated death domain (FADD). The interaction between TRIM21 and FADD enhances TRIM21 ubiquitin ligase activity to downregulate type I IFN by promoting the degradation of IRF7. But TRIM21 transcription is enhanced by type I IFN, suggesting TRIM21 plays an important role in a type I IFN negative feedback loop. TRIM21 also plays an important role in the regulation of NF-kb-dependent pro-inflammatory cytokine production through the negative regulation of NF-kb. Therefore, TRIM21 functions in both innate and acquired immunity through its E3 ligase activity. Recent reports suggest that it has a more direct intracellular antiviral capability. It was reported that the antiviral capacity of TRIM21 is through its Fc binding domain (Mallery et al., Proc Natl Acad Sci USA. 107(46): p. 19985-90). TRIM21 binds, with high affinity, to the Fc domain of immunoglobin, which are attached to the incoming virus, and target it to the proteasome via its E3 ubiquitin ligase activity. Rapid proteasomal degradation of virions in the cytosol occurs before translation of virally encoded genes can commence. Therefore, a dysregulation of TRIM21 could result in reduced antiviral clearance, as is often observed in CFS patients. Murine TRIM21 knockout mice appear phenotypically normal if left undisturbed, however; when challenged with TLR agonists they produce abnormally high levels of pro-inflammatory cytokines compared to wild-type mice (Espinosa et al., J Exp Med, 2009. 206(8): p. 1661-71). TRIM21 was originally identified as an autoantigen in Sjogren's syndrome and systemic lupus erythematosus. Both diseases have many overlapping symptoms to that of CFS such as chronic fatigue, inflammation, exercise intolerance, and muscle and joint pain and like CFS, diseases also occur to greater extent in women. Moreover, preliminary research suggests that the cancer drug Rituxan (rituximatab), which lowers the level of B cells, may be an effective treatment for a subgroup of CFS patients (Fluge and Mella, BMC Neurol, 2009. 9: p. 28) suggestive of an autoimmune condition similar to Sjogren's syndrome and systemic lupus. A defect in the TRIM21 pathway is consistent with an autoimmune condition characterized by the excessive production of pro-inflammatory cytokines, and the hyper-reactivity of IFN-α as is often observed in CFS patients.

Plasmacytoid dendritic cells are the primary producers of type I IFN; they are responsible for over 95% of type I IFN produced by leukocytes. Although they have the ability to produce all type I IFNs, the primary product of plasmacytoid dendritic cells is IFN-α. Large quantities of IFN are produced by pDCs in response to viral infection through the initiation of pattern recognition receptors known as Toll-like receptors (TLR). Type I IFN producing TLRs of pDCs are located in endosomal compartments and are activated by ssRNA (TLR7/8) and by CpG dsDNA (TLR9). IFN then proceeds to act locally and globally, through the activation of the interferon-alpha/beta receptors, IFNAR1 and IFNAR2. The binding of IFN to its receptor results in subunit dimerization followed by activation of their associated Janus protein kinases, which in turn phosphorylate several proteins, including STAT1 and STAT2.

A number of clinical observations are consistent with a pDC involvement in CFS. First, pDC are found primarily in the gut, the spleen and the lymph nodes. Therefore, a pDC involvement of CFS is consistent with the lymphadenopathy, splenomegaly and gastrointestinal abnormalities commonly reported in CFS patients. Second, the most prevalent inflammatory cytokines identified herein, IL-8, IL-6, TNF-α, MIP-α and MIP-1β are produced by pDCs; however, cytokines not produced by pDCs such as IL-1a, IL-2, IL-3, IL-4, IL-5, IL-13 and IL-15 are seldom upregulated in CFS patients. Finally, pDCs are responsible for 95% of all IFN-α production. Therefore, a dysregulation of IFN-α is most likely to occur in pDCs. These clinical and biochemical observations support that a TRIM21 dsyregulation occurs in the pDCs of CFS patients.

Production of type I interferon is involved with the innate antiviral response in CFS patients. During infection IFN-α promotes the production of IL-15, which performs a critical role in the development, maintenance and function of NK cells and activation of T cells. IFN-α also stimulates NK cell activity via the upregulation of tumor necrosis factor-related apoptosis-inducing ligand (TRAIL). Additionally, the three principal components of the RNase L antiviral pathway (OAS, RNase L and PKR) are transcriptionally upregulated by type I IFN. CFS literature is replete with references to NK cell and RNase L dysfunction. Dysregulation of type I IFN may contribute to the innate immune abnormalities associated with CFS.

Thus, initial low levels of INF-a combined with high levels of pro-inflammatory cytokines produced by pDC may set the stage for chronic inflammation, interferon hyper-reactivity and susceptibility to viral infection commonly observed in CFS patients.

Identified herein are various cytokine expression signatures that are reliably and reproducibly associated with CFS symptoms in subjects infected with a retorvirus. These cytokine expression signatures are different from that in healthy subjects who are not infected with a retrovirus, or who do not display CFS symptoms, as described herein. It is presently thought that infection with a retrovirus can induce changes in cytokine expression patterns. Significant changes in cytokine expression can cause inflammation within a subject's body. When cytokine expression in an infected individual becomes significantly different from that in an uninfected individual, it is thought that the associated inflammation can cause symptoms of neuroimmune diseases. In some instances, the symptoms can be one or more symptoms of CFS. In other instances, the inflammatory responses can cause neuroimmune diseases other than CFS, such as fibromyalgia, myalgic encephalitis, atypical multiple sclerosis, autism, non-epileptic seizures, or Gulf War Syndrome.

Physical symptoms of CFS can include, but are not limited to, those described in Carruthers et al. 2003 J Chronic Fatigue Syndrome 11, 1-12. More specifically, physical symptoms can include post-exertional malaise or fatigue, sleep dysfunction, and pain; have two or more neurlogical/cognitive manifestations and one or more symptoms from two or the categories of autonomic, neuroendocrine and immune manifestations. Autonomic manifestations can include orthostatic intolerance-neurally mediated hypotension (NMH); postural orthostatic tachycardia syndrome (POTS); delayed postural hypotension; light-headedness, extreme pallor; nausea and irritable syndrome; urinary frequency and bladder dysfunction; palpitations with or without cardiac arrhythmias; or exertional dyspnea. Neuroendocrine manifestations can include loss of thermostatic stability-subnormal body temperature and marked diurnal fluctuation; sweating episodes; recurrent feelings of feverishness and cold extremities; intolerance of extremes of heat and cold; marked weight change-anorexia or abnormal appetite; loss of adaptability and worsening of symptoms with stress. Immune manifestations can include tender lymphnodes, recurrent sore throat, recurrent flu-like symptoms, general malaise, new sensitivities to food, or medications or chemicals. In order to meet the criteria for CFS, these symptoms will have persisted for at least six months and usually have a distinct onset, although onset may be gradual.

The above proposed mechanism may explain why some subjects (such as subject 2623, infra) can be diagnosed as infected with a retrovirus but do not appear to display any, some, or all symptoms of a neuroimmune disease. In such a subject, a cytokine expression signature may not be significantly different enough from the cytokine expression pattern in an uninfected individual. Therefore, no or substantially no chronic inflammation occurs, and there are no or substantially no apparent symptoms of neuroimmune disease.

The above-proposed mechanism may also accommodate the changes in symptoms sometimes seen in a subject with chronic neuroimmune diseases. It is reasonable to assume that, over time, fluctuation of specific inflammatory cytokine expression levels can occur. Such fluctuation in expression of a cytokine could reasonably lead to a fluctuation in one or more symptoms of a neuroimmune disease.

Diagnosis

A cytokine expression signature, as described herein, can be used to diagnose a retroviral infection, conditions associated with a retroviral infection, or a neuroimmune disease. For example, a cytokine expression signature, as described herein, can be used to diagnose a retroviral infection in a subject. As another example, a cytokine expression signature, as described herein, can be used to diagnose a neuroimmune disease in a subject.

A cytokine expression signature used to diagnose a retroviral infection, conditions associated with a retroviral infection, or a neuroimmune disease can include level, activity, or expression of a cytokine as described herein. A cytokine of an expression signature and level, activity, or expression relative to a control can be as discussed above.

A method of diagnosis can include determination of level, activity, or expression of one or more cytokines of a cytokine expression signature in a subject or a sample of a subject. The cytokine level, activity, or expression signature profile (e.g., the expression pattern of cytokines of the signature) can be correlated with the presence of a retrovirus in the subject or the sample of the subject. Correlation of the cytokine expression signature and presence of a retrovirus can serve as, or contribute to, the diagnosis of a retroviral infection in the subject. Similarly, the cytokine level, activity, or expression signature profile (e.g., the expression pattern of cytokines of the signature) of a subject or a sample of the subject can be correlated with a neuroimmune disease in the subject. Determination in a subject or a sample of a subject of a cytokine level, activity, or expression signature correlated to a neuroimmune disease can serve as, or contribute to, the diagnosis of the neuroimmune disease in the subject.

Sample and Subject

Methods for the identification of a cytokine level, activity, or expression signature described herein are generally performed on a subject or on a sample from a subject. A sample can contain or be suspected of containing a retrovirus.

A sample can be a biological sample from a subject. A sample can be a blood sample, a serum sample, a plasma sample, a cerebrospinal fluid sample, or a solid tissue sample. For example, the sample can be a blood sample, such as a peripheral blood sample. As another example, a sample can be a solid tissue sample, such as a prostate tissue sample. A sample can include cells of a subject. For example, a sample can include cells such as fibroblasts, endothelial cells, peripheral blood mononuclear cells, hematopoietic cells, or a combination thereof.

The subject can be a subject having, diagnosed with, suspected of having, or at risk for developing a retroviral infection. A subject considered at risk of developing a retroviral infection can be, for example and without limitation, an individual with a familial history of the retrovirus, an individual contacted with a biological sample suspected of comprising the retrovirus, or an individual residing in a region comprising a cluster of individuals with the retroviral infection.

The subject can be a subject having, diagnosed with, suspected of having, or at risk for developing a neuroimmune disease or a lymphoma. For example, a subject can have, be diagnosed with, be suspected of having, or be at risk for developing a retroviral-related neuroimmune disease or a retroviral-related lymphoma. For example, a subject can be tested for the presence of an retrovirus where the subject exhibits one or more sign or a symptom associated with a neuroimmune disease or a lymphoma. As another example, a subject can have been diagnosed with a neuroimmune disease or lymphoma, or diagnosed with a retroviral-related neuroimmune disease or retroviral-related lymphoma.

A subject considered at risk of developing a neuroimmune disease or lymphoma can be, for example and without limitation, an individual with a familial history of a neuroimmune disease or lymphoma or an individual residing in a region comprising a cluster of individuals with a neuroimmune disease or lymphoma. For example, a subject can be considered at risk of developing CFS, if, without limitation, the individual has a familial history of CFS, or the individual resides in a region comprising a cluster of individuals with CFS.

In some cases, subjects infected with a retrovirus can exhibit no or substantially no persistent symptoms; i.e., they are apparently healthy. In other cases, subjects infected with a retrovirus are diagnosed with CFS. In other cases, subjects infected with a retrovirus are diagnosed with one or more cancer. In other cases, subjects infected with a retrovirus exhibit altered immune responses. In some cases, subjects infected with a retrovirus exhibit digestive-tract symptoms. Some subjects infected with a retrovirus develop multiple clinical symptoms, for example both CFS and cancer.

For example, a subject can be one which fulfills the 1994 CDC Fukuda Criteria for CFS (Fukuda et al., Ann Intern Med 1994; 121: 953-9); the 2003 Canadian Consensus Criteria (CCC) for ME/CFS (Carruthers et al, J Chronic Fatigue Syndrome 2003; 11:1-12; Jason et al., J Chronic Fatigue S 2004; 12:37-52), or both the Fukuda and CCC criteria. The CCC requires post-exertional malaise, which many clinicians believe is the sine qua non of ME/CFS. In contrast, the Fukuda and 1991 Oxford Criteria do not require exercise intolerance for a diagnosis of ME/CFS. The CCC further requires that subjects exhibit post-exertional fatigue, unrefreshing sleep, neurological/cognitive manifestations and pain, rather than these being optional symptoms.

The subject can be an animal subject, preferably a mammal, more preferably horses, cows, dogs, cats, sheep, pigs, mice, rats, monkeys, guinea pigs, and chickens, and most preferably a human.

As another example, the subject can be an animal, such as a laboratory animal that can serve as a model system for investigating a neuroimmune disease or lymphoma (see e.g., Chen, R. et al., Neurochemical Research 33: 1759-1767, 2008; Kumar, A., et al., Fundam. Clin. Pharmacol. 23(1): 89-95, February 2009; Gupta, A., et al, Immunobiology 214: 33-39, 2009; Singh, A., et al., Indian J. Exp. Biol. 40: 1240-1244, 2002; Ford, R. J., et al. Blood 109: 4899-4906, 2007; Smith, M. R., et al., Leukemia 20: 891-893, 2006; Bryant, J., et al., Lab. Invest. 80: 557-573, 2000; M′kacher, R., et al., Cancer Genet Cytogenet. 143: 32-38, 2003).

Device

Also provided is a device for use in detecting a cytokine expression signature described herein. Such a device can detect one of more cytokines or cytokine levels described herein. A device as described herein can be contacted with a biological sample so as to detect presence or level of one or more cytokines described herein.

Devices for detection of cytokines are understood in the art (see e.g., Khan et al. 2004 Cytometyery Part B: Clinical Cytometry 61B(A), 35-39; Li and Reichert 2003 Langmuir 19(5), 1557-1566; Huang et al. 2001 Analytical Biochemistry 294(1), 55-62; Haab 2005 Molecular and Cellular Proteomics 4, 377-383; Luchansky and Bailey 2010 Anal Chem 82(5), 1975-1981; Elshal and McCoy 2006 Methods 38(4), 317-323; Cytokine Antibody Array, Isogen Life Science, Netherlands; BioPlex Cytokine Assay, Bio-Rad; xMAP, Luminex Corp. Austin, Tex.; Human Cytokine Array Kit, R&D Systems, Minneapolis, Minn.). One of ordinary skill in the art can adapt conventional cytokine-detection devices for specificity with respect to one or more cytokines described herein. A device can incorporate a predictive algorithm described herein. A device can include an indicator for when a combination of cytokines of an specified expression signature described herein is present in a sample. A device can include an indicator for when a combination of levels of cytokines of an specified expression signature described herein is present in a sample.

A device can include an array (e.g., a microarray) for detection of one of more cytokines or cytokine levels described herein. A device can include a cytokine array membrane created by spotting capture antibodies onto the membrane. For example, a device can provide high-throughput simultaneous screening of multiple cytokine expression based on a protein array system. For example, a device can include an antibody-based array for detection of one of more cytokines or cytokine levels described herein. For example, a device can include an silicon photonic microring resonator for real-time detection of one or more cytokines described herein on account of their spectral sensitivity toward surface binding events between a target and antibody-modified microrings (see generally, Luchansky and Bailey 2010 Anal Chem 82(5), 1975-1981). For example, a device can include a multiplex bead array cytokine assay (see generally, Elshal and McCoy 2006 Methods 38(4), 317-323). For example, a device can include a cytokine detection protein array that combines cDNA microarray technology and sandwich fluoroimmunoassay, where a protein array can be printed by spotting one or more cytokines described herein onto planar substrates (see generally Li and Reichert 2003 Langmuir 19(5), 1557-1566).

Therapeutic Methods

Also provided is a process of treating a retroviral infection or a neuroimmune disease in a subject. As described herein, a cytokine expression signature of a subject or a sample of a subject can be correlated to a retroviral infection, thus providing or contributing to a diagnosis of a retroviral infection in the subject. As described herein, a cytokine expression signature of a subject or a sample of a subject can be correlated to a neuroimmune disease, thus providing or contributing to a diagnosis of the neuroimmune disease in the subject. Upon detection or determination of a cytokine expression signature described herein, a subject can be diagnosed with a retroviral infection or a neuroimmune disease and thereafter administered appropriate therapeutic treatment.

Protocols or agents for treatment of a neuroimmune disease can be according to a conventional therapeutic treatment known in the art.

The neuroimmune disease being diagnosed or treated can be CFS. Treating CFS can comprise administration of a therapeutically effective amount of an agent that restores cytokine expression to that of a healthy individual, which restores cytokine expression to levels similar to those in a healthy individual, which restores cytokine signaling to that of a healthy individual, or which restores cytokine signaling to levels similar to those in a healthy individual. Treating CFS can suppress or prevent CFS symptoms.

Furthermore, the present disclosure provides methods of treating symptoms of a retroviral infection, or directly treating a retroviral infection, in a subject. Protocols or agents for treatment of a retroviral infection can be according to a conventional therapeutic treatment known in the art. Therapeutic agents for treatment of a retroviral infection include, but are not limited to, a retroviral integrase inhibitor (e.g., raltegravir, Merck & Co., brand name Isentress; L-000870812, Merck & Co.) and a nucleoside reverse transcriptase inhibitor (e.g., tenofovir disoproxil fumarate, Gilead Sciences, brand name Viread; zidovudine, GlaxoSmithKline, azidothymidine (AZT)) (see Singh et al. 2010 PLoS ONE 5(4): e9948).

Treating symptoms of a retroviral infection, or directly treating a retroviral infection, can comprise administration of a therapeutically effective amount of an agent that restores cytokine expression to that of a healthy individual, which restores cytokine expression to levels similar to those in a healthy individual, which restores cytokine signaling to that of a healthy individual, or which restores cytokine signaling to levels similar to those in a healthy individual. Treating symptoms of a retroviral infection, or directly treating a retroviral infection, can suppress or prevent retroviral infection symptoms.

In some embodiments, a therapeutic agent can be a cytokine antagonist. The cytokine antagonist can be an anti-cytokine antibody, such as an anti-IFNα antibody or an anti-IFNγ antibody (see, eg, Jkurkovich et al., Medical Hypotheses 59(6): 770-780, 2002, Anticytokine therapy—new approach to the treatment of autoimmune and cytokine-disturbance diseases). The cytokine antagonist can be an agent possessing anti-TNF properties, such as infliximab or etanercept. The cytokine antagonist can possess anti-interleukin-1 (IL-1) or anti-interleukin-6 (IL-6) properties. The cytokine antagonist can be a glucocorticoid.

Methods described herein are generally performed on a subject in need thereof. A subject in need of the therapeutic methods described herein can be diagnosed with a neuroimmune disease, such as CFS, or at risk thereof. A subject in need of the therapeutic methods described herein can be infected with a retrovirus, diagnosed with a retroviral infection, or exhibiting one or more symptoms of a retroviral infection. A determination of the need for treatment will typically be assessed by a history and physical exam consistent with the disease or condition at issue. Diagnosis of the various conditions treatable by the methods described herein is within the skill of the art. The subject can be an animal subject, preferably a mammal, more preferably horses, cows, dogs, cats, sheep, pigs, mice, rats, monkeys, guinea pigs, and chickens, and most preferably a human.

An effective amount of an agent described herein is generally that which can restore cytokine expression to that of a healthy individual, which restores cytokine expression to levels similar to those in a healthy individual, which restores cytokine signaling to that of a healthy individual, or which restores cytokine signaling to levels similar to those in a healthy individual. An effective amount of an agent can suppress or prevent some, substantially all, or all symptoms of a neuroimmune disease, such as CFS. Alternatively, an effective amount of an agent can suppress symptoms related to neuroimmune disease, such as CFS. Symptoms related to CFS can include those used to diagnose CFS as described herein.

When used in the treatments described herein, a therapeutically effective amount of an agent can be employed in pure form or, where such forms exist, in pharmaceutically acceptable salt form and with or without a pharmaceutically acceptable excipient. For example, the compounds of the invention can be administered, at a reasonable benefit/risk ratio applicable to any medical treatment, in a sufficient amount to suppress or prevent a retroviral infection, a neuroimmune disease, such as CFS, or altered cytokine expression that is associated with a retroviral infection or a neuroimmune disease, such as CFS.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where large therapeutic indices are preferred.

The specific therapeutically effective dose level for any particular subject will depend upon a variety of factors including the disorder being treated and the severity of the disorder; activity of the specific compound employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present invention will be decided by an attending physician within the scope of sound medical judgment.

Administration of an agent can occur as a single event or over a time course of treatment. For example, an agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed prior to, concurrent with, or after conventional treatment modalities for a retroviral infection or a neuroimmune disease, such as CFS.

An agent can be administered simultaneously or sequentially with another agent, such as an antibiotic, an antiinflammatory, or another agent. For example, an agent can be administered simultaneously with another agent, such as an antibiotic or an antiinflammatory. Simultaneous administration can occur through administration of separate compositions, each containing one or more of agent described herein, an antibiotic, an antiinflammatory, or another agent. Simultaneous administration can occur through administration of one composition containing two or more of an agent described herein, an antibiotic, an antiinflammatory, or another agent. An agent can be administered sequentially with an antibiotic, an antiinflammatory, or another agent. For example, an agent can be administered before or after administration of an antibiotic, an antiinflammatory, or another agent.

Compositions or agents described herein can be administered in a variety of means known to the art. For example, administration can be parenteral, pulmonary, oral, topical, intradermal, intramuscular, intraperitoneal, intravenous, subcutaneous, intranasal, epidural, ophthalmic, buccal, or rectal administration. As another example, administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents will be known to the skilled artisan and are within the scope of the invention.

General

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

This example describes methods that can be used to obtain nucleic acid samples from subjects.

DNA and RNA isolation. Whole blood can be drawn from subjects by venipuncture using standardized phlebotomy procedures into 8-mL greencapped Vacutainers containing the anti-coagulant sodium heparin (Becton Dickinson). Plasma can be collected by centrifugation, aspirated and stored at −80° C. for later use. The plasma can be replaced with PBS and the blood resuspended and further diluted with an equal volume of PBS. PBMCs can be isolated by layering the diluted blood onto Ficoll-Paque PLUS (GE Healthcare), centrifuging for 22 min at 800 g, aspirating the PBMC layer and washing it once in PBS. The PBMCs (approximately 2×10⁷ cells) can be centrifuged at 500 g for 7 min and either stored as frozen unactivated cells in 90% FBS and 10% DMSO at −80° C. for further culture and analysis or resuspended in TRIzol (Invitrogen) and stored at −80° C. for DNA and RNA extraction and analysis. DNA can be isolated from TRIzol according the to manufacturer's protocol and also can be isolated from frozen PBMC pellets using the QIAamp DNA Mini purification kit (QIAGEN) according to the manufacturer's protocol and the final DNA can be resuspended in RNase/DNase free water and quantified using the Quant-iT™ Pico Green dsDNA Kit (Invitrogen). RNA can be isolated from TRIzol according to the manufacturer's protocol and quantified using the Quant-iT Ribo Green RNA kit (Invitrogen). cDNA can be made from RNA using the iScript Select cDNA synthesis kit (Bio-Rad) according to the manufacturer's protocol.

### Example 2

This example describes methods of amplifying, and determining the nucleic acid sequence of, XMRV polynucleotides.

PCR. Nested PCR can be performed with separate reagents in a separate laboratory room designated to be free of high copy amplicon or plasmid DNA. Negative controls in the absence of added DNA can be included in every experiment. Identification of XMRV gag and env genes can be performed by PCR in separate reactions. Reactions can be performed as follows: 100 to 250 ng DNA, 2 μL of 25 mM MgC12, 25 μL of HotStart-IT FideliTaq Master Mix (USB Corporation), 0.75 μL of each of 20 μM forward and reverse oligonucleotide primers in reaction volumes of 50 μL. For identification of gag, 419F (5′-ATCAGTTAACCTACCCGAGTCGGAC-3′) (SEQ ID NO: 5) and 1154R (5′-GCCGCCTCTTCTTCATTGTTCTC-3′) (SEQ ID NO: 6) can be used as forward and reverse primers. For env, 5922F (5′-GCTAATGCTACCTCCCTCCTGG-3′) (SEQ ID NO: 7) and 6273R (5′-GGAGCCCACTGAGGAATCAAAACAGG-3′) (SEQ ID NO: 8) can be used. For both gag and env PCR, 94° C. for 4 min initial denaturation can be performed for every reaction followed by 94° C. for 30 seconds, 57° C. for 30 seconds and 72° C. for 1 minute. The cycle can be repeated 45 times followed by final extension at 72° C. for 2 minutes. Six microliters of each reaction product can be loaded onto 2% agarose gels in TBE buffer with 1 kb+DNA ladder (Invitrogen) as markers. PCR products can be purified using Wizard SV Gel and PCR Clean-Up kit (Promega) and sequenced. PCR amplification for sequencing full-length XMRV genomes can be performed on DNA amplified by nested or semi-nested PCR from overlapping regions from PBMC DNA. For 5′ end amplification of R-U5 region, 4F (5′-CCAGTCATCCGATAGACTGAGTCGC-3′) (SEQ ID NO: 9) and 1154R can be used for first round and 4F and 770R (5′-TACCATCCTGAGGCCATCCTACATTG-3′) (SEQ ID NO: 10) can be used for second round. For regions including gag-pro and partial pol, 350F(5′-GAGTTCGTATTCCCGGCCGCAGC-3′) (SEQ ID NO: 11) and 5135R (5′-CCTGCGGCATTCCAAATCTCG-3′) (SEQ ID NO: 12) can be used for first round followed by second round with 419F and 4789R (5′-GGGTGAGTCTGTGTAGGGAGTCTAA-3′) (SEQ ID NO: 13). For regions including partial pol and env region, 4166F (5′-CAAGAAGGACAACGGAGAGCTGGAG-3′) (SEQ ID NO: 14) and 7622R (5′-GGCCTGCACTACCGAAAT TCTGTC-3′) (SEQ ID NO: 15) can be used for first round followed by 4672F (5′-GAGCCACCTACAATCAGACAAAAGGAT-3′) (SEQ ID NO: 16) and 7590R (5′-CTGGACCAAGCGGTTGAGAATACAG-3′) (SEQ ID NO: 17) for second round. For the 3′ end including the U3-R region, 7472F (5′-TCAGGACAAGGGTGGTTTGAG-3′) (SEQ ID NO: 18) and 8182R (5′-CAAACAGCAAAAGGCTTTATTGG-3′) (SEQ ID NO: 19) can be used for first round followed by 7472F and 8147R (5′-CCGGGCGACTCAGTCTATC-3′) (SEQ ID NO: 20) for second round. The reaction mixtures and conditions can be as described above except for the following: For larger fragments, extension can be done at 68° C. for 10 min instead of 72° C. All second round PCR products can be column purified as mentioned above and overlapping sequences can be determined with internal primers. Nested RT-PCR for gag sequences can be done as described with modifications. GAG-O-R primer can be used for 1st strand synthesis; cycle conditions can be 52° C. annealing, for 35 cycles. For second round PCR, annealing can be at 54° C. for 35 cycles.

Once nucleic acids have been amplified by PCR, standard sequencing techniques can be used to determine the nucleic acid sequence thereof. Standard in silico translation techniques can be used to determine amino acid sequences from nucleic acid sequences.

### Example 3

Cytokine analysis can be made by any quantitative method including but not limited to microplate assays such as Enzyme-linked immunosorbent assay (ELISA); multiplexing assay using antibody-conjugated microspheres, such as the Luminex xMAP Bead-based assay or Bender MedSystems bead-based system; systems involving the amplification of cytokine mRNA of the direct measurement of intracellular cytokines using flow cytometry or any other method that can quantitatively measure cytokines.

### Example 4

This example describes biomarkers associated with neuroimmune diseases, and specifically, with CFS. The methods in this example are as described in Examples 1-3, unless otherwise specified.

Cytokine and chemokine profiles are altered by infection. The inventors therefore examined the levels of 26 cytokines and chemokines from 156 XMRV-infected individuals and 140 healthy controls in an attempt to identify any hallmarks of XMRV infection. XMRV status was determined both by PCR-based and serological experiments, which detected XMRV env nucleic acid and protein, respectively.

Table 2 shows that a number of cytokines and chemokines are differentially expressed in XMRV-infected individuals. Notably, inflammatory chemokines such as IL-8 and MIP-1α and MIP-1β are upregulated in XMRV-infected subjects.

TABLE 2
Cytokines and chemokines up-regulated in XMRV-infected subjects

| | XMRV positive | | XMRV negative | | |
|---|---|---|---|---|---|
| | Mean | S.E. | Mean | S.E. | |
| IL-8 | 1067 | (267) | 11.1 | (1.3) | <0.0001 |
| MIP-1β | 1840 | (580) | 157 | (40) | <0.0001 |
| TNF-α | 109 | (48) | 12.8 | (4.6) | <0.0001 |
| IL-6 | 271 | (78) | 29 | (12) | <0.0001 |
| IL-2 | 99 | (59) | 29 | (11) | <0.0001 |
| IP-10 | 84 | (15) | 32.6 | (3.0) | <0.0001 |
| Eotaxin | 258 | (18) | 87.5 | (5.9) | <0.0001 |
| IL-12 | 272 | (18) | 210 | (34) | 0.0002 |
| Rantes | 26191 | (3554) | 8458 | (529) | 0.0041 |
| MCP-1 | 468 | (42) | 421 | (41) | 0.0003 |
| MIP-1α | 673 | (360) | 91 | (28) | 0.006 |

Table 3 shows that a number of cytokines were down regulated in XMRV-infected subjects when compared to healthy controls. Notably, IL-13, involved in anti-inflammatory responses, is down regulated in XMRV-positive subjects. IFN-α is also down regulated, as is IL-7, which is a key regulator of interferon signaling.

TABLE 3
Cytokines and chemokines down-regulated in XMRV-infected subjects

| | |
|---|---|
| XMRV positive | XMRV negative |

| | Mean | S.E. | Mean | S.E. | |
|---|---|---|---|---|---|
| IL-13 | 24.4 | (2.4) | 89.5 | (6.9) | <0.0001 |
| IL-5 | 7.11 | (0.64) | 22.2 | (5.3) | <0.0001 |
| IL-7 | 21.1 | (4.8) | 82 | (7.3) | <0.0001 |
| MIG | 43.7 | (7.3) | 83 | (13) | <0.0001 |
| IFN-α | 29.5 | (3.0) | 60.6 | (4.4) | <0.0001 |

Table 4 lists cytokines and chemokines that are differentially expressed in XMRV-infected subjects relative to healthy controls, and describes their functions.

TABLE 4
Cytokines and chemokines that are differentially expressed in
XMRV-positive and -negative subjects

| | | |
|---|---|---|
| Cytokine/ | | |
| Chemokine | P value | Function In Inflammation |

Upregulated in XMRV-infected subjects

| | | |
|---|---|---|
| IL-6 | <0.0001 | Stimulates chronic inflammation |
| MIP-1α | 0.0062 | Elevated in neurodegenerative disease |
| IL-8 | <0.0001 | RNase L and CMV activated |
| MIP-1β | <0.0001 | Elevated in Neurodegenerative disease |
| TNF-α | <0.0001 | Stimulates chronic inflammation |
| MCP-1 | 0.003 | Elevated in chronic inflammatory diseases |

Down regulated in XMRV-infected subjects

| | | |
|---|---|---|
| IL-13 | <0.0001 | Inhibits inflammatory cytokine production |
| IL-7 | <0.0001 | Stimulates proliferation of B and T lymphocytes |
| | | and NK cells |
| IFN-α | <0.0001 | Stimulates macrophages and NK cells to elicit an |
| | | anti-viral response |
| GM-CSF | <0.0001 | Stimulates proliferation of B and T lymphocytes |
| | | and NK cells |

### Example 5

This example describes a method of predicting a subject's XMRV status. Unless otherwise described, methods are as described in Examples 1-4.

Using data described above, the present inventors have developed an algorithm that predicts XMRV infection status from chemokine and cytokine expression information, with about 95% accuracy (Table 5). The inventors used the data described, including the pre-determined XMRV status, above as a training set for a Random Forest algorithm. The prediction algorithm was constructed using a standard Random Forest learning algorithm.

TABLE 5
Accuracy of Random Forest algorithm in predicting XMRV status

| Actual | | | Control | Positive |
|---|---|---|---|---|
| Class | Total Cases | Percent Correct | N = 137 | N = 159 |
| Control | 140 | 92.857 | 130 | 10 |
| Positive | 156 | 95.513 | 7 | 149 |

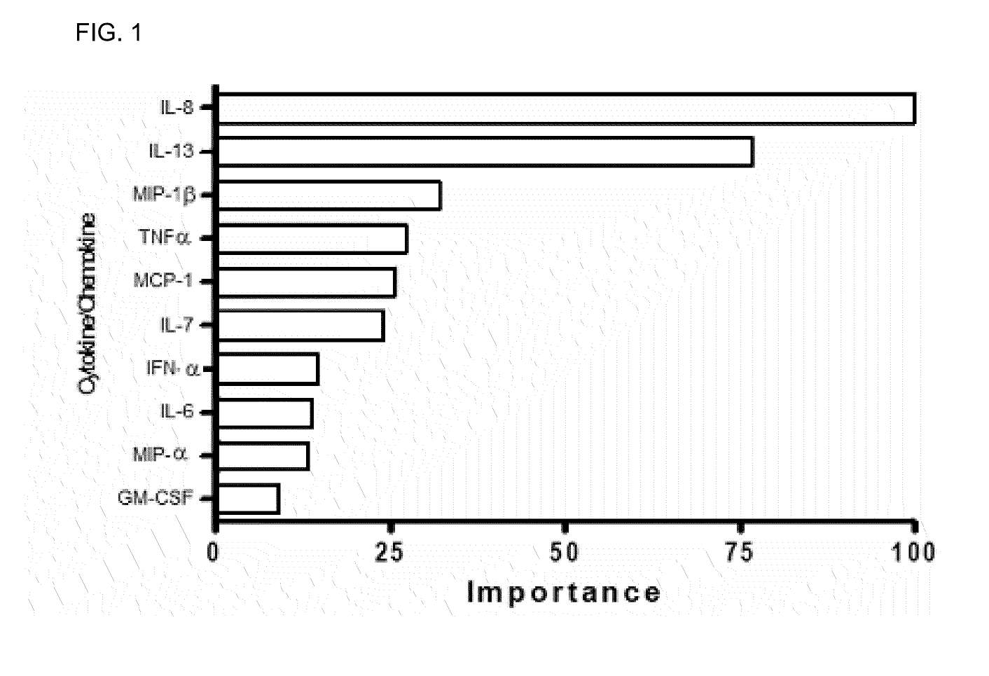

The Random Forest prediction algorithm identified the cytokines and chemokines listed in Table 4, above, as most critical in identifying XMRV status of an individual. FIG. 1 shows the relative importance of each.

When individual cytokines and chemokines are assigned a value of importance such that IL-8 is 100, IL-13 is 90, MIP-1β is 80, TNF-α is 70, MCP-1 is 60, IL-7 is 50, IFN-α is 40, IL-6 is 30, MIP-1α is 20, and GM-CSF is 10 then the prediction of CFS can be made by any combination of cytokines, cytokines and chemokines or chemokines with a combined value of about 210 or greater.

### Example 6

This example describes a method of identifying XMRV-infected subjects. Unless otherwise described, methods are as described in Examples 1-5.

For some individuals in the dataset used in these experiments, data was available which indicated the presence of γδ T-cells. γδ T-cells are cells that play an active role in the regulation and resolution of pathogen-induced immune responses. They accumulate at sites of inflammation caused by infections; and also in auto-immune diseases. γδ T-cells are also known to up-regulate MIP1-α, MIP1-β, and TNF-α. Clinically, the presence of γδ T-cells indicates chronic infection or cancer.

Data was collected from subjects who had been diagnosed with CFS who were subsequently diagnosed with cancer. Many of the CFS patients were also γδ T-cell positive patients; and all CFS patients subsequently tested were found to have XMRV. These results are summarized in Table 6.

TABLE 6
γδ T-cells can be detected in CFS subjects with cancer

| ID# | XMRV status | γδ T-cell status | Type of Cancer |
|---|---|---|---|
| 1103 | positive | positive | MCL |
| 1109 | positive | negative | Thymoma |
| 1125 | positive | positive + IGH | MCL |
| 1186 | positive | positive | Lymphoma |
| 1199 | positive | positive | Lymphoma |
| 1150 | positive | positive | Lymphoma |
| 1320 | positive | Not tested | Thymoma |
| 1321 | Not tested | Not tested | MCL |
| 1174 | positive | positive | Thymoma |
| 1205 | positive | Not tested | lymphoma |
| 1172 | positive | positive | MCL |
| 1127 | positive | positive | CLL |
| 1322 | Not tested | Not tested | MCL |
| 1181 | positive | Not tested | CLL |
| 1188 | positive | positive | CLL |
| 1189 | positive | positive | MCL |

subjects labeled as “Not Tested” were deceased by the time subsequent data collection for XMRV/T-cell status occurred
MCL = mantle cell lymphoma;
CLL = chronic lymphocytic leukemia

### Example 7

This example describes the phenotype of XMRV-infected subjects. Unless otherwise described, methods are as in Examples 1-6.

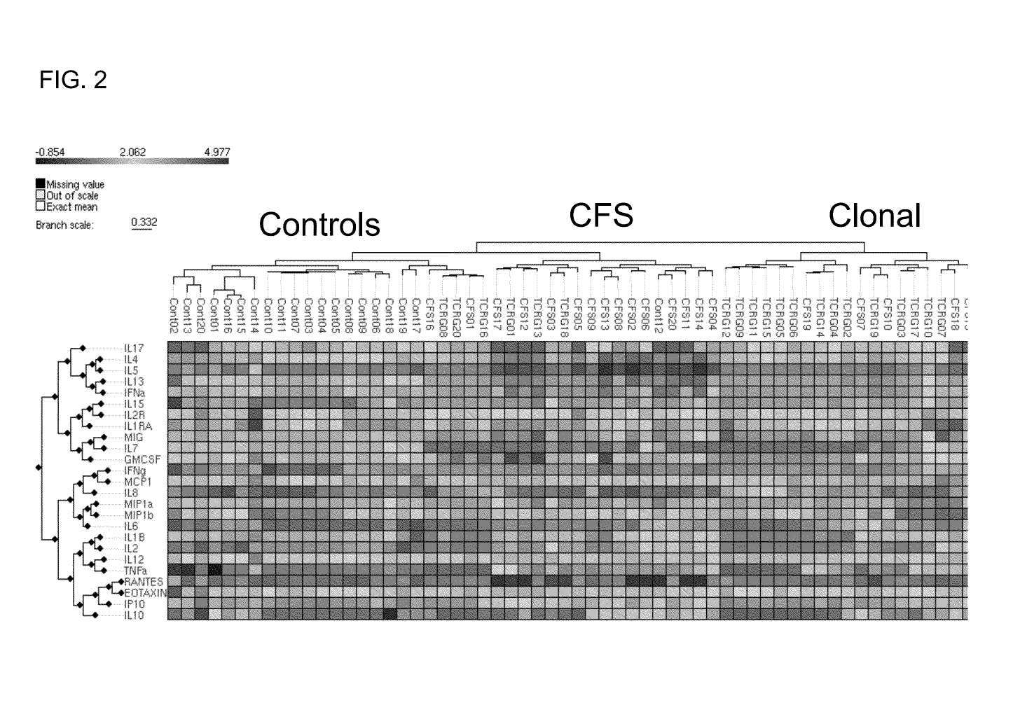

Clustering analysis was applied to the cytokine/chemokine dataset as described above. Cluster analysis clearly identified three groups that include healthy controls; CFS patients that have elevated γδ T-cell populations, and CFS patients who do not have elevated γδ T-cell populations (see e.g., FIG. 2). The γδ T-cell positive group has a prominent inflammatory response, as indicated by the high expression of pro-inflammatory cytokines and chemokines. Without being limited by theory, the present inventors hypothesize that this inflammation contributes to or causes the CFS symptoms associated with XMRV pathology. The inventors also hypothesize that inflammation may be a marker of disease progression.

### Example 8

This example describes the cytokine signature and disease state of an XMRV-positive subject. Methods are as in examples 1-7, unless otherwise specified.

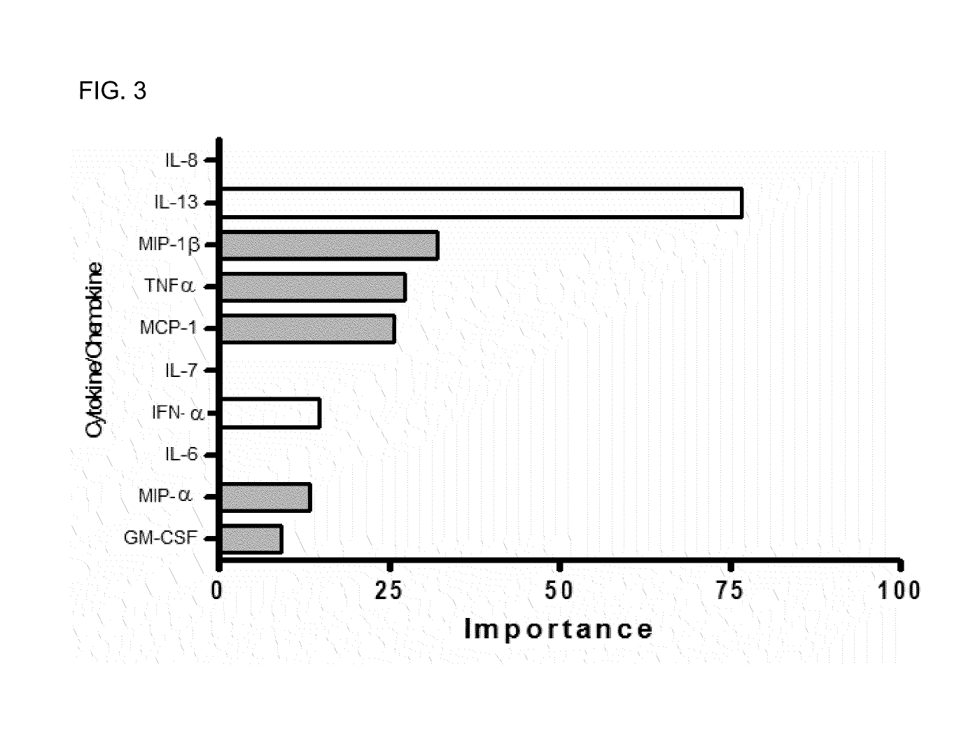

Subject 2623 is a 52-year-old female. She is positive for XMRV as determined by PCR and seroconversion tests, but does not display symptoms of chronic immune disease. FIG. 3 shows the expression levels of the cytokines and chemokines that were identified by the Random Forests analysis (supra) as a signature of XMRV infection. The cytokines and chemokines that were in the normal range have been removed from this dataset (eg, IL-8, IL-7 and IL-6). Of the remaining cytokines and chemokines, IL-13 and IFN-α show decreased expression relative to that in an uninfected subject; whereas MIP1α, MIP1β, TNFa and GM-CSF show increased expression relative to that in an uninfected subject. Not shown is subject 2623's increased IL-12 expression; IL-2 expression was identified by the cluster analysis (supra) to be important in staging disease progression.

### Example 9

This example describes the cytokine signature and disease state of an XMRV-positive subject. Methods are as in examples 1-8, unless otherwise specified.

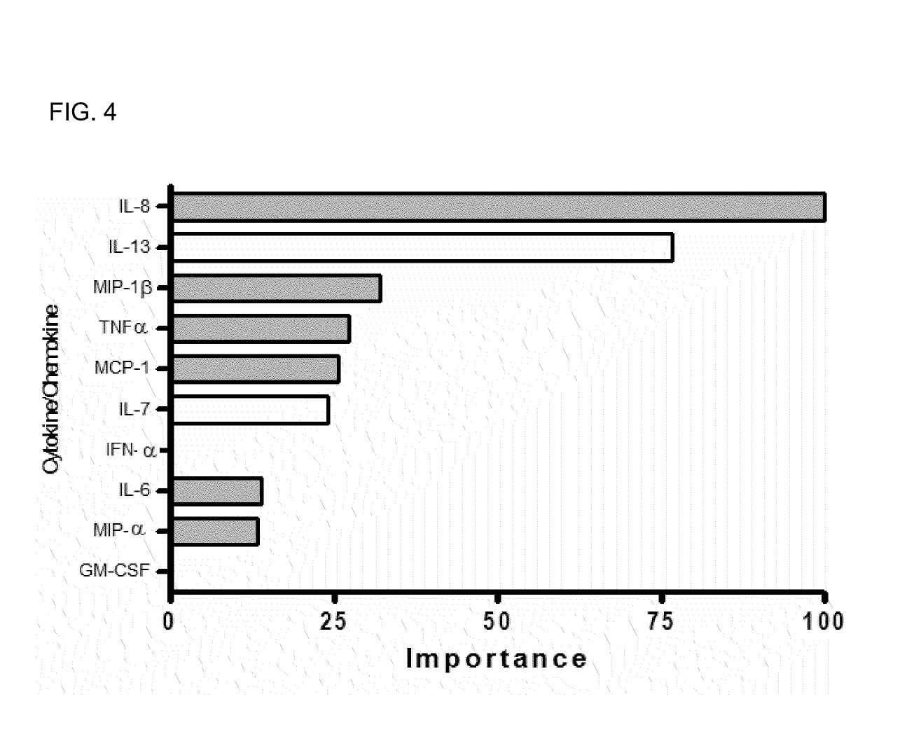

Subject 1127 is a 63-year-old female. She is positive for XMRV and has been diagnosed with CFS. She also has clonal populations of γδ-T cells, and eventually developed CLL. FIG. 4 shows the expression levels of the cytokines and chemokines that were identified by the Random Forests analysis (supra) as a signature of XMRV infection. The cytokines and chemokines that were in the normal range have been removed from this dataset (eg, IFN-α, GM-CSF).

### Example 10

This example describes the cytokine signature and disease state of an XMRV-positive subject. Methods are as in examples 1-9, unless otherwise specified.

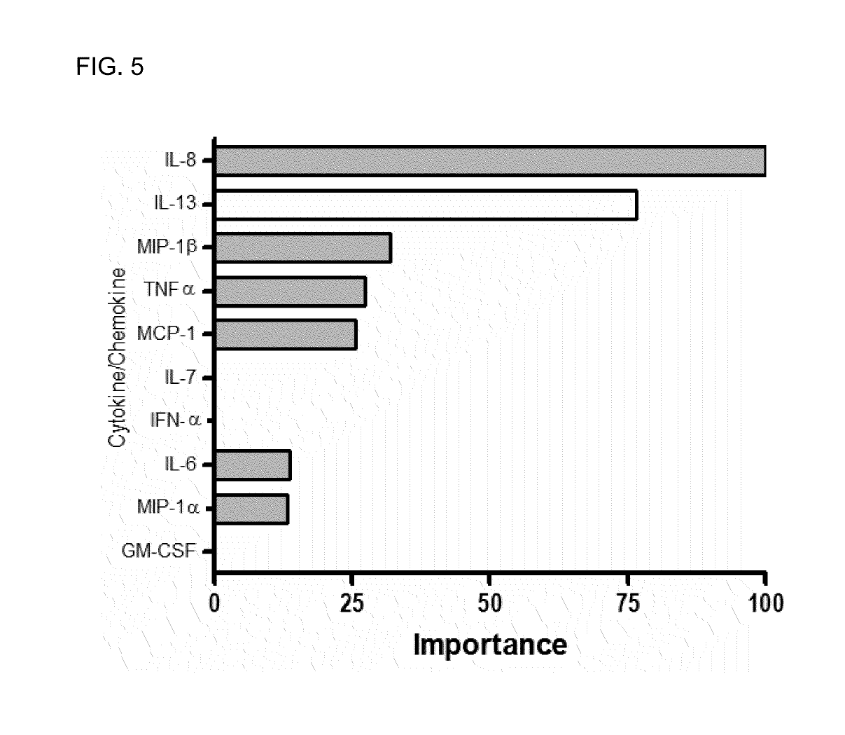

Subject 967 is a 31-year-old female. She is positive for XMRV and has chronic immune disease. She does not have clonal populations of γδ-T cells. FIG. 5 shows the expression levels of the cytokines and chemokines that were identified by the Random Forests analysis (supra) as a signature of XMRV infection. She has elevated IL-8, MIP-1α, MIP-1β, TNFα, IL-6 and GM-CSF. Not shown is subject 967's elevated RANTES levels; RANTES is not included as part of the diagnostic signature but is consistent with the findings of the cluster analysis.

### Example 11

This example describes cytokine and chemokine dysregulation in CFS patients. Methods are as in examples 1-10, unless otherwise specified.

CFS patients that meet both the CDC and Canadian Consensus Criteria. Patients were not selected on the basis of absence or presence of a known retroviral infection. Detection of cytokines was according to Multiplex Bean Immunoassays by patient and control.

Results showed an upregulation of the pro-inflammatory cytokines IL-6, IL-8, MIP-1α, MIP-1β and TNF-α in the plasma of CFS patients (Table 7).

TABLE 7

| | | | |
|---|---|---|---|
| | Patient | | |
| Patient Mean | Median | Control Mean | Control Median |
| N = 164 | N = 164 | N = 139 | N = 139 |
| (pg/mL) | (pg/mL) | (pg/mL) | (pg/mL) |

Up-Regulated

| | | | | |
|---|---|---|---|---|
| IL-8 | (8290 ± 1011) | 3574 | (13.1 ± 1.6) | 8.3 |
| IL-6 | (2623 ± 515) | 30 | (28.4 ± 10.7) | 4.0 |
| IL-1β | (219.3 ± 29.2) | 80.4 | (88.9 ± 20.5) | 55.8 |
| MIP-1β | (3701 ± 797) | 281 | (157.3 ± 40.3) | 85.0 |
| MIP-1α | (1813 ± 334) | 97 | (90.6 ± 19.2) | 63.9 |
| EOTAXIN | (205.4 ± 15.1) | 141.0 | (102.5 ± 8.5) | 84.1 |
| TNF-α | (158.1 ± 38) | 19.9 | (13.2 ± 4.25) | 6.3 |
| MCP-1 | (788.8 ± 51.3) | 593.1 | (423.8 ± 40.5) | 291.1 |
| IP-10 | (110.0 ± 20.8) | 34.3 | (35.6 ± 3.71) | 23.2 |
| IFN-γ | (20.0 ± 1.23) | 15.6 | (13.9 ± 0.866) | 11.8 |
| IL-12 | (215.4 ± 14.0) | 160 | (212.8 ± 31.1) | 131.8 |
| IL-2 | (30.6 ± 9.2) | 13.2 | (28.5 ± 10.2) | 11.8 |

Down-regulated

| | | | | |
|---|---|---|---|---|
| IL-13 | (38.27 ± 3.2) | 25.08 | (84.8 ± 6.5) | 77.5 |
| IL-7 | (57.2 ± 15) | 22.8 | (76.9 ± 6.8) | 68.4 |
| IFN-α | (48.1 ± 5.7) | 27.9 | (58.3 ± 4.1) | 48.7 |
| MIG | (54.7 ± 10.4) | 30.9 | (78.5 ± 11.6) | 53.2 |

All mean values are significant at the 95% C.I. by the log transformed Student t-Test.

CFS patients often report gastrointestinal issues similar to that of Crohn's disease and ulcerative colitis. In this study, pDCs from Crohn's patients, ulcerative colitis patients, and healthy controls were isolated and cultured in the presence or absence of the TLR agonist's imiquimod and ODN 2218.

Results showed that pro-inflammatory cytokine production of pDCs was significantly greater in the two patient groups than in the control group. Additionally, the upregulated cytokines observed, were similar to that observed in the plasma of CFS patients. These similarities suggest that CFS patients may also have dysfunction of pDCs.

To explore this possibility, pDCs were isolated from two healthy controls and one classic CFS patient who reported a viral flu-like onset of CFS and who has consistently displayed elevated plasma levels of pro-inflammatory cytokines. The isolated pDCs were cultured in the presence and absence of imiquimod and ODN for 22 hours. Levels of multiple cytokines were evaluated in culture media by multiplex analysis. Cytokine levels were determined in supernatants of pDC collected from CFS patients and healthy controls. pDC fraction of PBMC was isolated using CD304 positive selection (Miltenyi). TLR7 and 9 agonists were used to stimulate pDC for 22 hours. At the end of stimulation, supernatants were analyzed by Luminex multiplex assay.

Results showed little or no difference was observed for IL-1RA, IL-2, IL-2R, IL-4, IL-5, IL-7, IL-13, IL-17, IFN-γ GM-CSF, MIG, IP-10 and RANTES. IL-8 was elevated in all samples, including the non-stimulated pDCs, suggesting that activation occurred during the pDC purification process. But similar to the observed results in Crohn's disease and ulcerative colitis, a dramatic upregulation of the cytokines IL-6, MIP-1α, MIP-1β and TNF-α was observed in the pDCs isolated from the CFS patient but not in the control samples (data not shown). The stimulated pDC cytokine production was similar between the healthy controls; however, on average, the CFS patient's pDCs produced 3 times the inflammatory cytokines as the healthy controls. Strikingly, the IFN-a production of activated pDCs of the CFS patient was approximately 20 times that of the healthy controls in spite of having relatively normal plasma IFN-α levels (data not shown). Both control subjects had plasma cytokine levels within normal ranges, and the CFS subject had elevated plasma levels of pro-inflammatory cytokines, consistent with previous results (Data not show).

These data support that pDCs of CFS patients are more responsive to TLR agonists, with the greatest difference observed in the production IFN-α.

### Example 12

It is thought that an interrelated dysregulation occurs in the pathways mediating type I IFN and pro-inflammatory cytokine production in pDCs of CFS. Dysregulation of pDCs may account for the aberrant IFN and pro-inflammatory cytokine production as well as the other abnormalities observed in the innate immune system of CFS patients.

Previous data indicate that CFS patients have decreased plasma levels of INF-a. Because pDC are major producers of INF-a, it is expected that pathogenesis of CFS may be explained by dysfunction of these cells. Indeed, data demonstrated that while producing limited amount of INF-a in vivo, pDC from CFS are releasing 20 folds more INF-a when stimulated with TLR ligands in vitro as compared to healthy donors. Although the pattern of pro-inflammatory cytokine produced by stimulated pDC was similar between patients and controls, actual production was 3-20 folds higher in the CFS patients.

An ex vivo cell model system is used to characterize the mechanism of dysregulation of IFN and pro-inflammatory cytokines associated with CFS

IRF7, TRAF6, TRIM21 and FADD are evaluated at the level of transcription, translation and protein turnover (half-life) in pDCs cells of CFS patients and healthy controls in the presence and absence of TLR agonists. Sequencing and quantitative PCR, western blot analysis, ELISA of cell culture media; and IHC staining at multiple time points are used in the presence and absence of TLR agonists imiquimod and ODN 2213, using magnetically purified pDCs from CFS patients and healthy controls.

The ubiquidation, phosphorylation and nuclear translocation of IRF7, TRAF6, TRIM21 and FADD are characterized in pDCs of CFS patients and healthy controls in the presence and absence of TLR agonists.

Ten CFS patients that meet both the CDC and Canadian Consensus Criteria and 10 healthy controls are used in these experiments. Leukocytes are separated from whole blood by density gradient using Ficoll-Paque. Subject pDCs are purified by negative selection, in order to prevent any unforeseen effects by antibody binding, using the antibodies CD3, CD7, CD16, CD19, CD56, CD123, and CD235a (Miltenyi Biotec). The isolated pDCs are CD303 (BDCA-2)⁺, CD304 (BDCA-4/Neuropilin-1)⁺, CD123⁺, CD4⁺, CD45RA⁺, CD141 (BDCA-3)^{dim} and CD1c (BDCA-1)⁻, CD2⁻, which lack expression of lineage markers (CD3, CD14, CD16, CD19, CD20, CD56), and express neither myeloid markers such as CD13 and CD33, nor Fc receptors such as CD32, CD64, or FcαRI (Dzionek et al., Hum Immunol, 2002. 63(12): p. 1133-48). Cell line purity is evaluated by flow cytometry with the surface makers CD303 and CD123. Isolated pDCs are cultured on RPMI complete media supplemented with IL-3 (Jones et al., Nat Med, 2008. 14(4): p. 429-36) in the presence or absence of imiquimod and ODN 2213. The same experiment is made using pDC depleted lymphocytes as a control. Primary cells are cultured and analyzed for cytokine production at four separate time points, T=0 hrs, 6 hrs, 22 hrs and 4 days. Culture media is collected at each time point and flash frozen for cytokine analysis using Luminex multi-plex bead system. All measurements are made in triplicate for each time point then averaged.

Transcription analysis is made on the time point that produce optimal cytokine production by collecting cells on TRIzol for mRNA according to the manufacture's instructions; cDNA synthesis and Q-PCR is performed using the Superscript III Platinum CellsDirect Two-step qRT-PCR Kit. Transcriptome analysis is made using an Illumina HISeq 1000 with 50 pb single end reads and confirmed by RT-PCR. Proteomic analysis is then made by conducting a contig blast and a human reference guided alignment. Nuclear translocation is investigated by IHC using anti-TRIM21, FADD, IRF7 and IRF8 antibodies. Protein turnover is measured by western blot analysis on cells treated with GolgiStop to prevent cytokine secretion (BD PharMigen) and compared relative to control values and reported as a percentage change. Glyceraldehyde-3-phosphate dehydrogenase is used as a housekeeping gene control as well as a control for all experiments. Characterization of ubiquidation and phosphorylation of IRF7, TRAF6, TRIM21 and TRAFF is made by western blot using anti-ubiquitin and anit-phos[ho antibodies, which are commercially available. Nuclear localization is made by IHC of fixed pDC cells with anti-IRF7, TRAF6, TRIM21 and TRAF6 antibodies.

To determine differences between patient and controls, common nonparametric data analysis is used. Numerical data is analyzed with the computer program Prism and Flow cytometry analysis is made using the computer program FloJo. Densitometry analysis of western blots is made using program Image Quant (GE Health Sciences). DNASTAR software is used for denovo assembly and transcript identification of data is produced by next generation transcriptome sequencing.

It is expected to identify the point of dysregulation of cytokine production in the pDCs of CFS patients. If a decrease in transcription is observed, this would indicate that the disruption is occurring between the TLR and the initiation of transcription by the transcription complex. If normal transcription is observed but translation is not or is reduced compared to controls this would indicate the protein translation machinery is involved. In the event that a dysregulation is not observed at the level of TRIM21, FADD, IRF7 or IRF8, a transcriptome wide comparison is conducted between the pDCs of patients and controls and between the pDC depleted PBMCs of patients and controls to identify any differences that may account for the cytokine dysregulation not explained either by the TRIM21 pathway or dysregulation of pDCs.

Taken together, initial low levels of INF-a combined with high levels of pro-inflammatory cytokines produced by pDC may set the stage for chronic inflammation, interferon hyper-reactivity and susceptibility to viral infection commonly observed in CFS patients.

What is claimed is:

1. A method of diagnosing a neuroimmune disease or a retroviral infection in a subject, the method comprising:
comparing a cytokine expression signature of a subject with a control, the cytokine expression signature comprising an expression level of at least three cytokines or chemokines selected from the group consisting of IL-8, IL-13, MIP-1β, TNF-α, MCP-1, IL-7, IFN-α, IL-6, MIP-1α, and GM-CSF;
diagnosing the subject with a neuroimmune disease or a retroviral infection where the cytokine expression signature of the subject comprises at least one of
(i) IL-8 expression of at least about 10-fold higher in the subject, as compared to the control;
(ii) IL-13 expression of at least about 5-fold lower in the subject, as compared to the control;
(iii) MIP-1β expression of at least about 10-fold higher in the subject, as compared to the control;
(iv) TNF-α expression of at least about 10- or more-fold higher in the subject, as compared to the control;
(v) MCP-1 expression of at least about 1.1-fold higher in the subject, as compared to the control;
(vi) IL-7 expression of at least about 5-fold lower in the subject, as compared to the control;
(vii) IFN-α expression of at least about 2-fold lower in the subject, as compared to the control;
(viii) IL-6 expression of at least about 10- or more-fold higher in the subject, as compared to the control;
(ix) MIP-1α expression of at least about 2-fold higher in the subject, as compared to the control; and
(x) GM-CSF expression of at least about 0.7-fold lower in the subject, as compared to the control.

2. The method of claim 1 for diagnosing a retroviral infection comprising diagnosing the subject with a retroviral infection where the cytokine expression signature of the subject comprises at least one of (i)-(x).

3. The method of claim 1 for diagnosing an neuroimmune disease comprising diagnosing the subject with an neuroimmune disease where the cytokine expression signature of the subject comprises at least one of (i)-(x).

4. The method of claim 1, comprising determining a cytokine expression signature of a subject.

5. A method of claim 1, wherein the neuroimmune disease is selected from the group consisting of chronic fatigue syndrome, fibromyalgia, myalgic encephalitis, atypical multiple sclerosis, non-epileptic seizures, Gulf War Syndrome and autism.

6. The method of claim 1, wherein the cytokine expression signature comprises an expression level of at least three, at least four, at least five, at least six, at least seven, at least eight, at least nine, or all of IL-8, IL-13, MIP-1β, TNF-α, MCP-1, IL-7, IFN-α, IL-6, MIP-1α, and GM-CSF.

7. The method of claim 1, comprising administering an effective amount of an agent for treatment of a retroviral infection or a neuroimmune disease to a subject diagnosed with a retroviral infection or a neuroimmune disease.

8. The method of claim 1, comprising:
adding a weighted value for a cytokine or chemokine (a) present in the cytokine signature and (b) having an expression level of at least one of (i), (ii), (iii), (iv), (v), (vi), (vii), (viii), (ix), or (x), to arrive at a sum of weighted values; and
diagnosing the subject with a neuroimmune disease or a retroviral infection where the sum of weighted values is about 190 or greater, about 200 or greater, about 210 or greater, about 220 or greater, about 230 or greater, about 240 or greater, or about 250;
wherein the weighted value is selected from the group consisting of IL-8 is 100, IL-13 is 90, MIP-1β is 80, TNF-α is 70, MCP-1 is 60, IL-7 is 50, IFN-α is 40, IL-6 is 30, MIP-1α is 20, and GM-CSF is 10.

9. The method of claim 8, comprising diagnosing the subject with a neuroimmune disease or a retroviral infection where the sum of weighted values is about 210 or greater.

10. The method of claim 1, wherein the retroviral infection comprises an XMRV infection.

11. The method of claim 1, wherein the cytokine expression signature is determined from a culture of plasmacytoid dentritic cells (pDCs) isolated from the subject.

12. The method of claim 1, comprising determining a cytokine expression signature from a biological sample of the subject.

13. The method of claim 12, wherein the biological sample comprises a blood sample, a serum sample, a plasma sample, a cerebrospinal fluid sample, or a solid tissue sample.

14. The method of claim 12, wherein the biological sample comprises a serum sample or a plasma sample.

15. A device for detecting a cytokine expression signature of a subject comprising an array, wherein the array detects the presence or expression level at least three cytokines or chemokines selected from the group consisting of IL-8, IL-13, MIP-1β, TNF-α, MCP-1, IL-7, IFN-α, IL-6, MIP-1α, and GM-CSF.

16. The device of claim 15, wherein the array detects expression level of at least three of:
(i) IL-8 expression of at least about 10-fold higher in the subject, as compared to the control;
(ii) IL-13 expression of at least about 5-fold lower in the subject, as compared to the control;
(iii) MIP-1β expression of at least about 10-fold higher in the subject, as compared to the control;
(iv) TNF-α expression of at least about 10- or more-fold higher in the subject, as compared to the control;
(v) MCP-1 expression of at least about 1.1-fold higher in the subject, as compared to the control;
(vi) IL-7 expression of at least about 5-fold lower in the subject, as compared to the control;
(vii) IFN-α expression of at least about 2-fold lower in the subject, as compared to the control;
(viii) IL-6 expression of at least about 10- or more-fold higher in the subject, as compared to the control;
(ix) MIP-1α expression of at least about 2-fold higher in the subject, as compared to the control; and
(x) GM-CSF expression of at least about 0.7-fold lower in the subject, as compared to the control.
